(19) United States
(12) Patent Application Publication
(10) Pub. No.: US 2026/0166257 A1
(43) Pub. Date: Jun. 18, 2026

(54) METHOD AND DEVICE FOR PROVIDING MISSION FOR IMPROVING COGNITION OF DEMENTIA PATIENT

(71) Applicant: SEVENPOINTONE INC., Seoul (KR)

(72) Inventor: Hyeonjun LEE, Seoul (KR)

(73) Assignee: SEVENPOINTONE INC., Seoul (KR)

(21) Appl. No.: 19/399,849

(22) Filed: Nov. 25, 2025

(30) Foreign Application Priority Data
Dec. 18, 2024 (KR) 10-2024-0190145

(51) Int. Cl. A61M 21/00; G16H 20/70

(52) U.S. Cl. CPC A61M 21/00; G16H 20/70; A61M 2021/005; A61M 2205/3303

(57) ABSTRACT

The present disclosure relates to a method and device for providing a mission for improving cognition of a dementia patient. The device includes a memory configured to store various mission data; and a processor configured to collect data of a target dementia patient through an artificial intelligence call, identify a dementia status of the target dementia patient based on the collected data, generate first mission data according to the dementia status of the identified target dementia patient, transmit the first mission data to at least one of a dementia patient terminal or a caregiver terminal, obtain feedback data on the first mission data from at least one of the dementia patient terminal or the caregiver terminal, analyze whether the target dementia patient's cognition is improved based on the feedback data, and transmit the analysis result to at least one of the dementia patient terminal or the caregiver terminal.

### CROSS-REFERENCE TO RELATED APPLICATIONS

A claim for priority under 35 U.S.C. § 119 is made to Korean Patent Application No. 10-2024-0190145 filed on Dec. 18, 2024 in the Korean Intellectual Property Office, the entire contents of which are hereby incorporated by reference.

### BACKGROUND

### 1. Technical Field

The present disclosure relates to a method and device for providing a mission for improving cognition of a dementia patient.

### 2. Description of Related Art

Alzheimer's Disease (AD) is a brain disorder that occurs with aging, and caused by brain abnormalities that lead to the gradual decline of memory. Furthermore, AD can lead to dementia, a persistent and pervasive decline in cognitive function that interferes with daily life. The cognitive function refers to various intellectual abilities, including memory, language, visuospatial awareness, judgment, and abstract thinking, each of which is closely related to specific areas of the brain.

Mild cognitive impairment (MCI) refers to a condition in which memory and cognitive function decline compared to age-matched individuals, but not yet dementia. Mild cognitive impairment can progress to Alzheimer's disease, making early detection and intervention crucial for early detection.

The dementia (or MCI) test typically involves selective tests, diagnostic tests, and differential tests. The selective tests are conducted at regional dementia support centers, public health centers, and other locations. However, these tests require in-person visits from test individuals, and the fact that they are conducted by individuals poses challenges in terms of validity and efficiency. Specifically, the individuals who can visit a test center in person are highly unlikely to have dementia, reducing the effectiveness of the test. Furthermore, in-person test can take dozens of minutes per individual, resulting in significant time and cost losses.

The telephone dementia test is a phone-based dementia test technology that utilizes artificial intelligence (AI) technology, and according to the test, instead of a human, AI conducts phone calls with target individuals of the dementia test.

### SUMMARY

The present disclosure is to provide a system that can improve cognition of a dementia patient by providing a mission.

Furthermore, the present disclosure is to provide a system that can provide a mission appropriate for the various conditions of dementia patients.

In an aspect of the present disclosure, a device for improving cognition of a dementia patient may include a memory configured to store various mission data; and a processor configured to communicate with the memory and exchanging data, wherein the processor is configured to: conduct a dementia test on the dementia patient through an artificial intelligence call and collect a result of the dementia test, obtain a dementia status of the dementia patient based on the result of the dementia test, wherein the dementia status includes a dementia attribute, a dementia degree, and a dementia type, select first mission data for improving cognition of the dementia patient based on the dementia status, and transmit the first mission data to at least one of a dementia patient terminal or a caregiver terminal, receive feedback data on the first mission data from at least one of the dementia patient terminal or the caregiver terminal, when conducting the dementia test, analyze usage patterns of applications installed on the dementia patient terminal and selectively provide a type of the dementia test, and based on a use frequency of a news application of the dementia patient being high of among the applications, conduct the dementia test on topics related to current affairs, and collect location information of the dementia patient terminal to identify a frequency of going out or a change in location of the dementia patient, and determine the dementia patient's behavior based on a determined result to adjust a difficulty of the dementia test related to cognitive function, wherein the attribute includes an age, a gender, an education level, a residence, a hometown, a presence of spouse, an income level, a presence of allergy, and a use of walking aid, and wherein the processor is further configured to: when selecting the first mission data, select a mission that reflects the interests and activity preferences of the gender, select the mission considering a problem type and a difficulty level based on the education level, select the mission that reflects environmental differences between urban and rural areas based on the residence, select the mission that stimulates memories related to the hometown of the dementia patient based on the hometown, select the mission that is performed with the spouse based on the presence of the spouse, select the mission considering a cost required to perform the mission based on the income level, select the mission to avoid an allergen while performing the mission based on the allergy, select the mission to limit a movement distance of the dementia patient and an activity type based on the use of walking aid, based on the dementia type being Alzheimer's disease, select the mission that stimulates memory, based on the dementia type being vascular dementia, select the mission that repeats words presented on a screen and the mission that expresses emotions, based on the dementia type being Lewy body dementia, select the visual recognition mission to identify and identify a specific object in a displayed image or the mission to listen to music and then state a title, based on the dementia type being frontotemporal dementia, select at least one mission to record a conversation with the caregiver and then submit voice data, the mission to verbally describe facial expression displayed on the screen, and the mission to submit a photo after completing a housework, and based on the dementia type being Parkinson's disease dementia, select at least one of the mission of rolling a ball with a hand and taking a picture, the mission of recording a conversation with the caregiver and submitting the record, or the mission of submitting a picture after small muscle exercise.

In another aspect of the present disclosure, a method for improving cognition of a dementia patient, performed by an electronic device may include conducting a dementia test on the dementia patient through an artificial intelligence call and collecting a result of the dementia test; obtaining a dementia status of the dementia patient based on the result of the dementia test, wherein the dementia status includes a dementia attribute, a dementia degree, and a dementia type; selecting first mission data for improving cognition of the dementia patient based on the dementia status, and transmitting the first mission data to at least one of a dementia patient terminal or a caregiver terminal; receiving feedback data on the first mission data from at least one of the dementia patient terminal or the caregiver terminal; when conducting the dementia test, analyzing usage patterns of applications installed on the dementia patient terminal and selectively providing a type of the dementia test, and based on a use frequency of a news application of the dementia patient being high of among the applications, conduct the dementia test on topics related to current affairs; and collecting location information of the dementia patient terminal to identify a frequency of going out or a change in location of the dementia patient, and determine the dementia patient's behavior based on a determined result to adjust a difficulty of the dementia test related to cognitive function, wherein the attribute includes an age, a gender, an education level, a residence, a hometown, a presence of spouse, an income level, a presence of allergy, and a use of walking aid, and wherein the electronic device is configured to: when selecting the first mission data, select a mission that reflects the interests and activity preferences of the gender, select the mission considering a problem type and a difficulty level based on the education level, select the mission that reflects environmental differences between urban and rural areas based on the residence, select the mission that stimulates memories related to the hometown of the dementia patient based on the hometown, select the mission that is performed with the spouse based on the presence of the spouse, select the mission considering a cost required to perform the mission based on the income level, select the mission to avoid an allergen while performing the mission based on the allergy, select the mission to limit a movement distance of the dementia patient and an activity type based on the use of walking aid, based on the dementia type being Alzheimer's disease, select the mission that stimulates memory, based on the dementia type being vascular dementia, select the mission that repeats words presented on a screen and the mission that expresses emotions, based on the dementia type being Lewy body dementia, select the visual recognition mission to identify and identify a specific object in a displayed image or the mission to listen to music and then state a title, based on the dementia type being frontotemporal dementia, select at least one mission to record a conversation with the caregiver and then submit voice data, the mission to verbally describe facial expression displayed on the screen, and the mission to submit a photo after completing a housework, and based on the dementia type being Parkinson's disease dementia, select at least one of the mission of rolling a ball with a hand and taking a picture, the mission of recording a conversation with the caregiver and submitting the record, or the mission of submitting a picture after small muscle exercise.

In still another aspect of the present disclosure, a system for improving cognition of a dementia patient may include a dementia patient terminal configured to provide mission data and obtain data related to perform a mission; a caregiver terminal configured to select or modify the mission data to be provided from the terminal of the dementia patient; and a computing device configured to provide mission data to at least one of the dementia patient terminal or the caregiver terminal, wherein the computing device is configured to: conduct a dementia test on the dementia patient through an artificial intelligence call and collect a result of the dementia test, obtain a dementia status of the dementia patient based on the result of the dementia test, wherein the dementia status includes a dementia attribute, a dementia degree, and a dementia type, select first mission data for improving cognition of the dementia patient based on the dementia status, and transmit the first mission data to at least one of a dementia patient terminal or a caregiver terminal, receive feedback data on the first mission data from at least one of the dementia patient terminal or the caregiver terminal, when conducting the dementia test, analyze usage patterns of applications installed on the dementia patient terminal and selectively provide a type of the dementia test, and based on a use frequency of a news application of the dementia patient being high of among the applications, conduct the dementia test on topics related to current affairs, and collect location information of the dementia patient terminal to identify a frequency of going out or a change in location of the dementia patient, and determine the dementia patient's behavior based on a determined result to adjust a difficulty of the dementia test related to cognitive function, wherein the attribute includes an age, a gender, an education level, a residence, a hometown, a presence of spouse, an income level, a presence of allergy, and a use of walking aid, and wherein the computing device is further configured to: when selecting the first mission data, select a mission that reflects the interests and activity preferences of the gender, select the mission considering a problem type and a difficulty level based on the education level, select the mission that reflects environmental differences between urban and rural areas based on the residence, select the mission that stimulates memories related to the hometown of the dementia patient based on the hometown, select the mission that is performed with the spouse based on the presence of the spouse, select the mission considering a cost required to perform the mission based on the income level, select the mission to avoid an allergen while performing the mission based on the allergy, select the mission to limit a movement distance of the dementia patient and an activity type based on the use of walking aid, based on the dementia type being Alzheimer's disease, select the mission that stimulates memory, based on the dementia type being vascular dementia, select the mission that repeats words presented on a screen and the mission that expresses emotions, based on the dementia type being Lewy body dementia, select the visual recognition mission to identify and identify a specific object in a displayed image or the mission to listen to music and then state a title, based on the dementia type being frontotemporal dementia, select at least one mission to record a conversation with the caregiver and then submit voice data, the mission to verbally describe facial expression displayed on the screen, and the mission to submit a photo after completing a housework, and based on the dementia type being Parkinson's disease dementia, select at least one of the mission of rolling a ball with a hand and taking a picture, the mission of recording a conversation with the caregiver and submitting the record, or the mission of submitting a picture after small muscle exercise.

Other specific details of the present disclosure are included in the detailed description and drawings.

### BRIEF DESCRIPTION OF THE FIGURES

FIG. 1 is a conceptual diagram of a dementia test device according to one embodiment of the present disclosure.

FIG. 2 is an exemplary diagram illustrating a dementia test process performed by a dementia test device according to one embodiment of the present disclosure.

FIG. 3 is a conceptual diagram illustrating a portion of an inbound request of a dementia testing device according to one embodiment of the present disclosure.

FIG. 4 is a flowchart schematically illustrating a portion of an inbound request-based dementia test method according to an embodiment of the present disclosure.

FIG. 5 is an exemplary diagram illustrating a STT multiplexing process in a dementia test device according to an embodiment of the present disclosure.

FIG. 6 illustrates a dementia patient cognitive improvement system according to an embodiment of the present disclosure.

FIG. 7 illustrates an example of a dementia patient cognitive improvement device according to an embodiment of the present disclosure.

FIGS. 8 to 11 are flow charts illustrating a method for improving cognition of a dementia patient according to an embodiment of the present disclosure.

FIG. 12 is an example of a user interface related to a mission provided to a dementia patient.

### DETAILED DESCRIPTION

The advantages and features of the present disclosure, and methods for achieving them, will become clear with reference to the embodiments described in detail below along with the accompanying drawings. However, the present disclosure is not limited to the embodiments disclosed below and may be implemented in various different forms. The present embodiments are merely provided to ensure that the present disclosure is complete, and provided to fully convey the scope of the present disclosure to those skilled in the art to which the present disclosure pertains. The present disclosure is defined only by the scope of the claims.

The terminology used herein is for the purpose of describing embodiments and is not intended to limit the disclosure. As used herein, singular forms also include plural forms, unless specifically stated otherwise in the context. As used in the specification, “comprises” and/or “comprising” does not exclude the presence or addition of one or more other elements in addition to the mentioned elements. Throughout the specification, the same reference numerals refer to the same elements, and “and/or” includes each and every combination of the elements mentioned. Although “first”, “second”, etc. are used to describe various elements, it is to be understood that these elements are not limited by these terms. These terms are merely used to distinguish one element from another. Accordingly, it should be understood that a first element mentioned below may also be a second element within the technical scope of the present invention.

In this specification, “AI call” refers to a phone that utilizes AI technology, where AI performs the call instead of a human. The AI call system uses Speech-to-Text (step STT) technology to convert the user's voice into text data and Text-to-Speech (TTS) technology to convert the text back into speech. The converted text data is analyzed using Natural Language Processing (NLP) technology to identify the user's intent and generate a corresponding response. In the field of dementia test, AI call may be used to automate test and determine the need for follow-up testing. This allows subjects to be tested over the phone without having to visit a testing institution in person, and automates the process of human examiners asking questions and recording answers, reducing testing time and costs. AI call may analyze test results and assist in identifying subjects requiring follow-up testing.

In this specification, “dementia test” refers to a dementia test using an AI call system. The AI Call System conducts the dementia test remotely via phone calls, eliminating the need for test subjects to physically visit the testing facility, and this may automate the questioning, recording, and analysis processes performed by human examiners, reducing testing time. The dementia test operates 24 hours a day and may process multiple tests simultaneously, ensuring efficient use of medical resources. The dementia test expands testing opportunities for older adults who have limited mobility or limited time, and may provide testing without geographical restrictions. AI may quantitatively analyze various linguistic characteristics, such as word count, transition scores, category scores, and pronation scores, to conduct objective assessments. The dementia test based on the AI Call System may analyze test results to identify individuals requiring follow-up testing and connect them to specialized medical institutions or dementia test centers.

In this specification, “language fluency value” refers to a numerical value of relevant abilities (e.g., semantic memory, executive function, working memory) used to assess the presence and/or progression of dementia. Quantitatively assessing a test subject's language ability plays a crucial role in assessing the likelihood of dementia. Dementia patients typically have impaired language abilities, which may manifest as: 1) a reduced number of words they may utter in a given period of time; 2) a tendency to use the same words repeatedly; and 3) frequent straying from the topic or giving irrelevant answers. Therefore, to obtain a “language fluency value”, the subject is asked to speak words related to a specific topic. The audio responses are converted into text data, and words relevant to the given topic are extracted.

The score is calculated based on factors such as the total number of words, the number of words in the first and second halves, the number of characters per word, the number of category changes, the number of words per category, and the number of duplicate words. The calculated language fluency score is compared to a pre-established reference value to determine the need for follow-up testing. The reference value is set for each testing group based on auxiliary information such as gender, age, education level, and number of people living in the same reference value.

The “language fluency value” plays an important role in dementia test based on the AI call system. 1) Shortened test time: Increased efficiency by reducing the test time to less than 3 minutes 2) Minimized target restrictions: Since it is not a paper-and-pencil test, there are almost no restrictions on the test subjects 3) Non-face-to-face testing: Testing may be conducted without the need for the tester and testee to meet face-to-face 4) Various instructions may be provided on the smartphone screen in the form of an app or web simultaneously with the call, allowing for accurate and easy testing anytime, anywhere.

“Language fluency” may be quantified using the following formula:

$$[ Mathematical Formula 1 ]$$

$$LFV = a 1 ⋆ A + a 2 ⋆ B + a 3 ⋆ C + a 4 ⋆ D + a 5 ⋆ E + a 6 ⋆ F + a 7 ⋆ G$$

LFV: Language Fluency Value
A: Total Word Count
B: First Half Word Count
C: Second Half Word Count
D: Average Character Count per Word
E: Number of Category Changes
F: Average Word Count per Category
G: Number of Duplicate Words
a1˜a7: Weighting Variables (satisfying a1+a2+a3+a4+a5+a6+a7=1, where a1˜a7 are respective constants greater than or equal to 0 and less than or equal to 1)
In an embodiment, the language fluency value may be calculated by reflecting not only the test subject's answers but also auxiliary information. Here, “auxiliary information” is basic information such as personal information about the test subject, and may include, for example, gender (male, female), age (teens, 20s, 30s, 40s, 50s, 60s, 70s, 80s, 90s, etc.), education level (elementary school graduate, middle school graduate, high school graduate, master's degree, bachelor's degree, doctorate, etc.), number of cohabitants (1 person, 2 people, 3 people, 4 people, etc.). A test content analysis module 145 may more accurately calculate the language fluency value by differentially applying the type, number, weight value, and the like of variables used to calculate the language fluency value based on auxiliary information such as age, gender, education level, and number of cohabitants.

Unless otherwise defined, all terms (including technical and scientific terms) used herein may be used with meanings commonly understood by those skilled in the art to which the present disclosure pertains. Furthermore, terms defined in commonly used dictionaries should not be interpreted ideally or excessively unless explicitly and specifically defined otherwise.

Hereinafter, embodiments of the present disclosure will be described in detail with reference to the attached drawings.

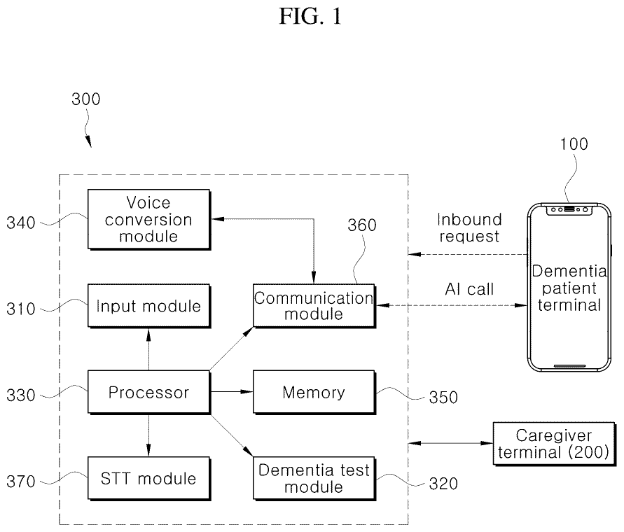

FIG. 1 is a conceptual diagram of a dementia test device according to one embodiment of the present disclosure.

As illustrated in FIG. 1, a computing device 300 according to one embodiment of the present disclosure may perform a dementia test by exchanging information with at least one of a dementia patient terminal 100 or a caregiver terminal 200, which includes a mobile device. The computing device 300 may include an input module 310, a dementia test module 320, a processor 330, a voice conversion module 340, a memory 350, a communication module 360, and an STT module 370. The computing device 300 of FIG. 1 may include the dementia test device.

The computing device 300 is a server that performs dementia test using the AI call. Here, the AI (Artificial Intelligence) call refers to a telephone using AI technology, where AI conducts the call instead of a human. Specifically, the AI call may provide a pre-stored voice consisting of an AI voice and/or a human-recorded voice to the connected terminal. For example, the pre-stored voice may include guidance, question voices, closing comments, and the like.

A user may participate in the dementia test by receiving the voice provided by the AI call and responding to the voice, thereby interacting with the AI. Meanwhile, in one embodiment, in the case that the AI call-based user and dementia test process is determined to be unsatisfactory, the pre-stored AI voice may be discontinued and a human may directly intervene to communicate with the dementia test participant, who is at least one user of the dementia patient terminal 100 and the caregiver terminal 200. In the case that the user believes the dementia test is not proceeding smoothly, the user may manually contact the dementia test service provider by pressing the “Call a Counselor” button displayed on the screen of at least one of the dementia patient terminal 100 or the caregiver terminal 200.

The computing device 300 may communicate with at least one of the dementia patient terminal 100 or the caregiver terminal 200. When a phone call is connected to an external device 20 of the user, the computing device 300 initiates an AI call and performs a voice question-and-answer-based dementia test. The computing device 300 analyzes the dementia test results, using voice responses obtained from at least one of the dementia patient terminal 100 or the caregiver terminal 200 during the test, to determine whether the user of the external device 20 qualifies for follow-up dementia testing. This process is performed automatically by the computing device 300 without direct intervention from a “human” such as a tester or administrator, significantly reducing the time and cost required for dementia testing. Furthermore, by preemptively filtering whether an in-person test is necessary via a phone call before the test subject visits the testing institution for testing, the effectiveness of follow-up dementia testing may be increased and the budget reduced.

The computing device 300 assesses the language fluency of the test subject through AI-based voice question-and-answer to determine whether follow-up testing is necessary. Specifically, the computing device 300 conducts a dementia test based on voice question-and-answer and analyzes the test subject's voice responses obtained therefrom to calculate a language fluency value, which quantifies language fluency. The dementia test device 10 compares and analyzes the calculated language fluency value with a preset value to determine whether follow-up testing is necessary. For example, in the case that the language fluency value is below the preset value, the user may be determined to be a subject for follow-up testing. In the case that the language fluency value is greater than or equal to the preset value, the user may be determined to be ineligible for follow-up testing. The dementia test performed by the computing device 300 may correspond to any one of three steps of testing including a selective test, a diagnostic test, and a differential test.

The input module 310 may collect first data containing a user's dementia test request to initiate a dementia test process using the computing device 300. Since the first data containing the dementia test request is generated by a user possessing at least one of the dementia patient terminal 100 or the caregiver terminal 200, if not properly controlled, excessive dementia test processes may cause server downtime or network paralysis. This may lead to budget waste and significantly impact dementia management projects. Therefore, an inbound dementia test device needs to precisely handle the “dementia test request” signal.

The dementia test process of the computing device 300 of the inbound type is initiated by the first data containing the dementia test request. The first data may be collected through one or more of the following methods: making a phone call, scanning a two-dimensional barcode, entering a web address, sending an SMS text message, tapping an app push button, touching an NFC tag, or entering a kiosk. The first data is typically generated by at least one of the dementia patient terminal 100, such as a smartphone, owned by a user wishing to participate in the dementia test, or the caregiver terminal 200. However, the first data may also be generated by a desktop PC, a kiosk, or a computer with an input device, and transmitted to the computing device 300. At least one of the dementia patient terminal 100 or the caregiver terminal 200 may be a mobile device owned by the testee, and may be a tablet PC, a desktop PC, a kiosk, or a computer with an input device. However, the device on which the dementia test process is performed needs to be a communication device equipped with a microphone.

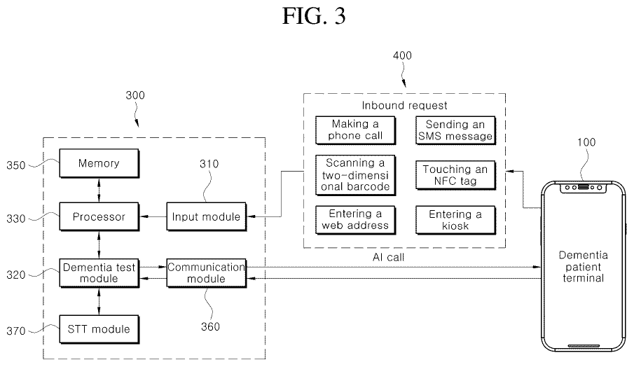

The first data, which includes the user's dementia test request, may be generated by one or more of the following methods: making a phone call, scanning a two-dimensional barcode, entering a web address, sending an SMS text message, tapping an app push button, touching an NFC tag, or entering a kiosk. From the perspective of the computing device 300, the “dementia test request” is received, and is therefore referred to as an “inbound request.” Referring to FIG. 3, the configuration of the inbound request 300 may be identified. A dementia test request generated by at least one of the dementia patient terminal 100 or the caregiver terminal 200 is transmitted to the computing device 300 via the input module 310 and serves as a trigger to initiate the dementia test process.

One embodiment of the first data collected through a phone call is as follows. When a user calls a designated phone number, the automatic response system (ARS) of the computing device 300 operates to collect the dementia test request. The ARS is included in the input module 310. The computing device 300 recognizes the user's voice command and, if necessary, prompts the user to input basic information via voice, or, if additional testing is required, allows the user to select a test by pressing a button.

One embodiment of the first data collected through two-dimensional barcode scanning is as follows. When a user scans a QR code with a smartphone, a webpage or app opens, and a dementia test request is received by the computing device 300 via the input module 310. After receiving the request, the computing device 300 automatically places a call to at least one of the dementia patient terminal 100 or the caregiver terminal 200 via the communication module 360. Once the call is connected, the dementia test process begins. During this process, the user's ID and initial information previously stored in the browser or smartphone are automatically entered via the QR code, allowing for rapid initiation of the dementia test process. Membership is not required, and the test may be conducted under a pseudonym. If the user consents to the request via the browser accessed via the QR code, one or more of the following secondary data may be collected: the user's name, phone number, gender, and highest level of education. Furthermore, personal information use consent information regarding the use of the secondary data may be obtained from the user.

One embodiment of the first data collected by entering a web address is as follows. A user may register a request by entering a pre-designated web address using a pre-installed web browser on an internet-connected computer, such as a smartphone or PC, and then accessing the dementia test page. The webpage provides basic information fields for users to directly enter their name, contact information, phone number, and desired test date. Based on the entered information, the computing device 300 automatically initiates a call to initiate the dementia test process.

In situations where the individual does not personally perform a test, such as when a parent over a certain age requests a dementia test, the user may pre-set the test information by entering the web address and notify the subject in advance, allowing family members to easily receive the test. Furthermore, the test information set in this manner may be sent to the subject in advance via a messenger app like KakaoTalk, facilitating easy sharing of information. Furthermore, the subject may adjust their schedule on the webpage to receive the test at a convenient time. This method of entering a web address offers the advantage of easy access even without a QR code.

One embodiment of the first data collected via SMS text message is as follows. When a user sends an SMS containing a specific code or keyword to a designated number, a dementia testing request is automatically accepted. The computing device 300 analyzes the content and sender number of the text message sent by the user to identify the user requiring testing. If the user is determined to require testing, a phone call is initiated to initiate the dementia testing process. If necessary, a reply text message may provide detailed testing procedures or a link. If the user includes a desired dementia testing date in the text message, the text message may be analyzed and a dementia testing phone call may be made on the desired date.

One embodiment of the first data collected via app push notification is as follows. If the computing device 300 has a dedicated dementia testing app connected to the Internet, the user may initiate a testing request via the app push notification. Clicking the push notification automatically transmits the user's identity information and basic health information to the app, and the testing begins immediately. The app-based method provides easy access for users who already have the app installed, and may support efficient testing by linking additional user information and records. Furthermore, the app allows users to check when they received a dementia test, what their dementia test scores were, and whether their scores are improving. This may be effective in long-term dementia patient management in many ways. Furthermore, it provides convenient information on dementia-friendly lifestyle and eating habits, various games and content for dementia prevention, and the locations of dementia support centers and medical institutions.

One embodiment of the first data collected by the NFC tag is as follows. When a user touches at least one of the dementia patient terminal 100 or the caregiver terminal 200, such as a smartphone, to the NFC tag at a specific location, a dementia test request is automatically collected. Recent advancements in NFC tag technology have enabled easy installation into thin media, such as stickers or printed paper. Most smartphones are equipped with readers that include sensors capable of recognizing NFC tags, allowing users to easily generate a dementia test request signal by simply touching the smartphone to the NFC tag. Particularly, since it's common to register the installation location when installing an NFC tag, it's easy to determine the user's location even when using at least one of the dementia patient terminal 100 or the caregiver terminal 200, which have location information like GPS turned off. Once the location information is determined, a customized test may be provided based on the starting location or situation of the dementia test.

One example of the first data collected through kiosk input is as follows. A user may directly request a dementia test through a kiosk installed in a hospital or public location. The kiosk is equipped with a touchscreen, allowing the user to press a request button, enter simple information, and then apply for the test. The kiosk method allows for on-site testing requests and allows the user to schedule a dementia test at a preferred time. Therefore, patients visiting the hospital for other medical treatments may easily schedule a dementia test, and accompanying caregivers may also schedule the test.

The dementia test module 320 may be included in or connected to the processor 330, receive and process data provided by the processor 330, and provide the processed value to the processor 330. The dementia test module 320 performs a dementia test on a subject who has at least one of the dementia patient terminal 100 or the caregiver terminal 200 connected to an artificial intelligence call for dementia test. The dementia test performed by the dementia test module 320 may be performed based on voice question and answer. For example, the dementia test module 320 may perform a dementia test by providing a question for the test to at least one of the dementia patient terminal 100 or the caregiver terminal 200, and obtaining a voice response of the subject from at least one of the dementia patient terminal 100 or the caregiver terminal 200. Specifically, the dementia test module 320 may provide a specific topic (or criterion, category, etc.) to the subject, and pre-set a word corresponding to the topic. A question voice requesting as many answers as possible during a given time period may be provided. The dementia test module 320 may acquire the response voices that the user responds to the question voices.

In one embodiment, the dementia test performed by the dementia test module 320 may include a first test and a second test that are performed sequentially. Here, the “first test” may be a practice test conducted prior to the second test to enhance the user's understanding of the test. In addition, the “second test” may be the main test that is actually used to determine the presence or absence of dementia or the level of dementia symptoms in the test subject.

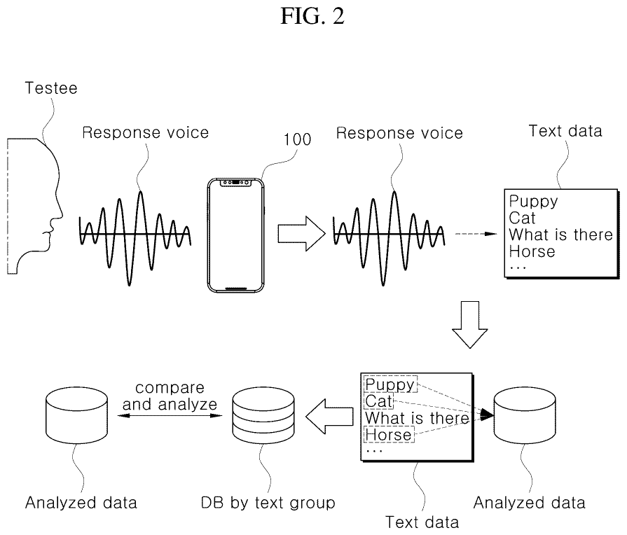

Details regarding the dementia test performed by the dementia test module 320 will be described below with reference to FIGS. 2 to 4.

The processor 330 may be configured with one or more processors. The processor 330 exchanges information with the input module 310, the dementia test module 320, the processor 330, the voice conversion module 340, the memory 350, the communication module 360, and the STT module 370, and may process data required for dementia test. One or more processors may be general-purpose processors such as a CPU, AP, or DSP (Digital Signal Processor), a graphics-only processor such as a GPU or VPU (Vision Processing Unit), or an artificial intelligence-only processor such as an NPU. One or more processors control the processing of input data according to predefined operation rules or artificial intelligence models stored in the memory.

The predefined operation rules or artificial intelligence models may be created through learning. Here, “created through learning” means that the basic artificial intelligence model is trained using a learning algorithm using a plurality of learning data, thereby creating predefined operation rules or artificial intelligence models configured to perform a desired characteristic (or purpose). This learning may be performed on the device itself, where the artificial intelligence according to the present disclosure is implemented, or through a separate server and/or system. Examples of learning algorithms include, but are not limited to, supervised learning, unsupervised learning, semi-supervised learning, or reinforcement learning.

The processor 330 collects and processes the second data, including at least one of the user's name, phone number, gender, or highest level of education, and personal information consent information regarding the use of the second data, along with first data, including the user's request for a dementia test, through the input module 310. In addition to the user's name, phone number, gender, and highest level of education, the second data may further include: date of birth, address, phone number, family history, current medical conditions, medication information, occupational history, lifestyle information, cognitive function history, language usage habits, social activity participation, and psychological state. To collect the second data, a separate questionnaire or game may be provided. To facilitate the collection of the second data, rewards may be provided for completing the questionnaire or game.

While collecting the second data is important for obtaining more accurate dementia test results, it often constitutes personal information. Therefore, consent for personal information may be obtained during the inbound requests, such as making a phone call, scanning a two-dimensional barcode, entering a web address, sending an SMS message, touching an app push notification, touching an NFC tag, or entering a kiosk.

The processor 330 analyzes information regarding at least one of the dementia patient terminal 100 or the caregiver terminal 200 from which the first data is generated and transmitted, and based on the external device information, the first data, and the second data, the processor 330 may configure a dementia test process tailored to the user's environment. One embodiment of at least one of the dementia patient terminal 100 or the caregiver terminal 200 from which the first data including the dementia test request is transmitted may include a mobile device such as a smartphone. After obtaining consent for personal information, the computing device 300 may collect location information from at least one of the dementia patient terminal 100 or the caregiver terminal 200 and determine whether the user, for whom location information is activated, frequently goes out or whether there are any changes in location at certain times.

In the case that location changes are minimal, the device may determine that the user has difficulty moving, and adjust the difficulty of the dementia test related to cognitive function. In the case that the user moves frequently, the device may further include a dementia test related to spatial awareness. Furthermore, by analyzing patterns such as the user's frequently used language or words, typing speed, and voice rate during calls, the device may identify language usage habits based on language patterns, and increase or decrease the difficulty of the dementia test based on frequently used words and context.

In addition, by analyzing whether the user frequently uses the smartphone or mainly uses it at certain times, the dementia test AI call may be avoided at times when the smartphone is frequently used or targeted at those times. Furthermore, the dementia test type may be selectively provided by analyzing the user's app usage pattern. By analyzing which apps, such as news, social media, messenger, and game apps, are primarily used, the dementia test process may be conducted based on topics related to current affairs for users who primarily use news apps, or the dementia test process may be provided for topics other than current affairs. For users who primarily use social media apps or messengers, the dementia test process may be conducted based on vocabulary or topics the user frequently uses or has expressed interest in on social media or messengers, or the difficulty of the dementia test may be adjusted by conducting the dementia test process on topics other than those topics.

The processor 330 may identify the location information of at least one of the dementia patient terminal 100 or the caregiver terminal 200 from which the first data is transmitted at the time of transmission, and based on the identified location information, may generate first jurisdictional information regarding the administrative district in which at least one of the dementia patient terminal 100 or the caregiver terminal 200 is located, and second jurisdictional information regarding the medical institution responsible for the location.

The processor 330 may subdivide the second jurisdictional information into metropolitan medical institutions and basic medical institutions, and based on the second jurisdictional information, may add guidance and additional examination processes for each medical institution to the voice question-and-answer-based dementia examination process provided by default to at least one of the dementia patient terminal 100 or the caregiver terminal 200.

The processor 330 may subdivide the user into metropolitan administrative agencies and basic administrative agencies based on the first jurisdictional information. Based on the first data, the second data, and the first jurisdictional information, the processor may analyze the number of dementia test requests, request methods, request locations, and request timing of the user's request. In the case that the same user requests dementia tests more than a certain number of times within a certain period, the processor may prevent the dementia test process from proceeding.

The processor 330 analyzes the response voice received through the communication module 360 in real time and separates the response voice into the user's voice and background voice. In the case that the background voice is determined to contain the voice of a person other than the user or to be generated in a place with noise levels exceeding 50 decibels, the processor may transmit a signal to at least one of the dementia patient terminal 100 or the caregiver terminal 200 regarding whether to stop the dementia test process.

To separate the user's voice and background voice in real time, a microphone array-based separation method and a neural network-based voice separation model may be used. In microphone array-based separation, multiple microphone arrays are used to isolate voice sources. Based on the location information of the sound source, sounds originating from a specific direction may be emphasized and sounds originating from other directions may be suppressed. For example, a user's voice coming from close to the smartphone microphone may be emphasized, while background sounds or voices may be suppressed. Furthermore, beamforming technology may be used to extract only the user's voice signal from a specific location, while noise or other people's voices originating from other directions may be separated as background sounds.

Neural network-based speech separation utilizes convolutional neural network (CNN), recurrent neural network (RNN), or transformer model to process voice data in real time and separate the user's voice from the background voice. Pre-trained speech separation models may also be used to separate the user's voice even in situations where multiple voice signals are mixed. This allows for real-time processing of the response voice signal, separating the user's voice from background noise into independent signals. To separate the user's response voice from background noise, a signal-to-signal correlation analysis method may be applied, which analyzes the frequency band of the voice signal to distinguish between the voice characteristics of the person speaking (e.g., voice pitch, pronunciation pattern) and background noise characteristics (e.g., irregular frequency bands) in real time.

The method for determining whether the background voice includes the voice of a person other than the user who is being tested for dementia is as follows. A speaker recognition algorithm is applied to distinguish the speaker from the voice signal. After pretraining the user's voice, it is possible to determine in real time whether a voice is from the same speaker. Since the first test is a practice dementia test, a speaker recognition algorithm may be applied to recognize the voice of the user who is a dementia test subject during the first test and to distinguish between the voices of the user who is a dementia test subject and those of others during the second test, which is the main test.

Furthermore, the volume, timbre, and pitch of the user's response voice during the first test may be analyzed to determine whether the user is a dementia test subject. In this way, the characteristics of each speaker's voice signal may be analyzed to determine the frequency and pitch of the voice. For example, cases where the user's voice characteristics and the background voice characteristics are clearly distinct may be configured as training data to separate the user's voice from the voice signals of other speakers. In the case that the background voice is determined to include the voice of a person other than the user, a signal indicating whether to stop the dementia test process may be transmitted to at least one of the dementia patient terminal 100 or the caregiver terminal 200 to obtain accurate dementia test results.

Determining whether background noise is generated in a location with noise levels exceeding 50 decibels may be accomplished by analyzing noise intensity using decibel measurements. The noise intensity, measured in decibels (dB), is measured from the response voice signal received by the communication module 360. To achieve this, a noise level meter function is implemented to calculate the average dB value of background noise collected over a certain period of time. In the case that the calculated noise level exceeds 50 decibels, the noise generated in that location is determined to be background noise affecting the user's voice signal. Frequency band analysis may also be used.

Background noise typically occurs in a specific frequency band. Therefore, this band may be analyzed in real time to establish a frequency range where noise levels exceeding 50 decibels are predominant. In the case that the signal intensity in a specific frequency band exceeds 50 decibels, the background noise is determined to be strong and may be recognized as noise that could affect the user's response. Determination may also be achieved using noise removal filtering technology and level monitoring. To remove background noise from a voice signal, a noise canceling filter is applied to extract only the noise component, and the decibel level of the filtered background noise signal may be monitored to determine whether there is noise exceeding 50 decibels. In the case that the background noise is determined to have been generated in a place where noise exceeds 50 decibels, an indication of whether to stop the dementia test process is transmitted to at least one of the dementia patient terminal 100 or the caregiver terminal 200, thereby allowing the dementia test to be performed in an environment where the user may obtain more accurate dementia test results.

The processor 330 may include the dementia test module 320 that receives the first data from the input module 310, connects a telephone call with at least one of the dementia patient terminal 100 or the caregiver terminal 200 for dementia testing, provides a guidance voice through the communication module 360, performs the first test, which is a preliminary practice test, to increase the test subject's understanding of the test process, and performs the second test, which is the main test used to determine the presence or absence of dementia disease or the level of dementia symptoms of the test subject.

The processor 330 analyzes the response voice by counting words, first half words, second half words, character count per word, number of category changes, word count per category, speaking speed, pronunciation accuracy, intonation, stress changes, voice tremors, topic consistency, maintenance of conversational flow, and number of duplicate words. The processor 330 may then apply an addition/deduction criterion to at least one of these factors to score the results of the dementia test process.

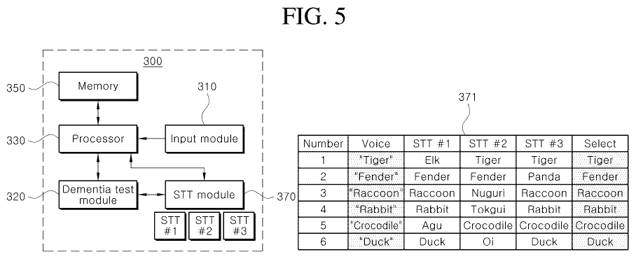

The processor 330 may control the STT module 370 including one or more STT models, convert the response voice into text by applying three or more STT models, compare the words in the converted text, and select a converted text that accounts for the majority of the converted text to score the results of the dementia test process. The word processing process of the STT module 370 is schematically illustrated in detail in FIG. 5.

The voice conversion module 340 may utilize various voice characteristics to provide a customized experience for users and may be converted into various voices to facilitate user interaction. 1) Voice conversion based on age is possible. The AI voice may be converted into different age groups, such as children, young adults, middle-aged, and elderly, to enable more natural and friendly communication with users. 2) Voice conversion based on gender is possible. The voice may be converted into male or female voices to suit user preferences. 3) Voice conversion based on emotion is possible.

The voice may be adjusted to sound calm, friendly, or encouraging depending on the situation. A calm tone may be used during questioning stages that require the user's focus, and an encouraging closing remark may be provided upon completion of the diagnosis, providing psychological stability. 4) Speed and intonation may be adjusted. To enhance the effectiveness of information delivery, difficult questions may be spoken slowly and clearly, while simple instructions may be delivered naturally and quickly. 5) The use of regional dialects is possible.

By providing regional dialects or familiar accents tailored to the user's linguistic background, more natural interactions may be achieved. Questions or guidance may be provided in the dialect of a specific region to build a sense of intimacy with users from that region. Particularly, using the voice conversion module 340 may convert not only questions but also the responses provided by the subject of the infringement diagnosis into standard Korean, resulting in more accurate infringement detection results. A regional dialect database is built to analyze responses, and dialects with the same meaning as the standard Korean are recognized as correct. Uninterpretable dialects are treated as noise.

Using the voice conversion module 340 in this way, the secondary data and the user's personal information are analyzed to provide a personalized, familiar voice pattern for each user, reducing resistance to the AI-based dementia test process and creating a comfortable environment, thereby increasing the completion rate of the dementia test.

The memory 350 stores at least one process for performing operations and stores the user's input information and data. A large amount of data may be stored in the memory, and a database system may be installed. To ensure smooth progress of the dementia test process, the personal information of users who have previously undergone dementia test may be stored, and a database of individuals in a specific age group (e.g., under 65 years of age) may be stored and managed. This database of individuals subject to test is transmitted to the processor 330, and to ensure efficient operation of the computing device 300, criteria may be provided to control dementia test only for individuals over a certain age group. In addition, the test results and test histories of individuals included in the database of individuals subject to test may be continuously updated, stored, and managed. By selectively sending AI calls only to individuals who actually require the test, excluding individuals with a recent test history, the sending of meaningless AI calls may be prevented. The computing device 300 manages the database of individuals subject to the test loaded in the memory 350 and may initiate AI call-based dementia test calls only for individuals who have not been tested within a preset period.

The communication module 360 performs a role in which the computing device 300 communicates with at least one of the dementia patient terminal 100 or the caregiver terminal 200. The communication module 360 provides the dementia test process by communicating with at least one of the dementia patient terminal 100 or the caregiver terminal 200, transmits a question voice to at least one of the dementia patient terminal 100 or the caregiver terminal 200, and receives an answer voice from at least one of the dementia patient terminal 100 or the caregiver terminal 200, thereby transmitting the result of the dementia test process. The communication module 360 may include at least one of a short-range communication module, a wired communication module, or a wireless communication module, and voice communication and data communication are possible at the same time. For example, the short-range communication module may include various short-range communication modules that transmit and receive signals using a wireless communication network at a short distance, such as a Bluetooth module, an infrared communication module, an RFID communication module, a WLAN communication module, an NFC communication module, and a Zigbee communication module. The wired communication module may include various wired communication modules such as a local area network (LAN) module, a wide area network (WAN) module, or a value-added network (VVAN) module, as well as various cable communication modules such as USB, HDMI, DVI, RS-232, power line communication, or POTS. The wireless communication module may include a wireless communication module that supports various wireless communication methods, such as a Wi-Fi module, GSM, CDMA, WCDMA, UMTS, TDMA, LTE, and 5G.

The STT module 370 refers to a module that converts voice into text. This will be described with reference to FIG. 5. The STT module 370 may include various STT engines, such as Google Speech-to-Text API, Microsoft Azure Speech API, IBM Watson Speech to Text, and Naver Clover Engine, for example. Each of these STT models has its own algorithm for converting speech into text. Based on the algorithm and learning data, it may provide different results. By configuring these multiple STT models, text conversion may be performed with higher accuracy even for speech containing specific pronunciations, intonations, or noises. The STT module 370 applies three or more STT models and compares the text results obtained from each model.

During this process, the text words generated by each STT model are listed to check for identical or similar results, and the words or phrases that account for the majority of the converted text are selected. For example, if two or more models produce identical words in the conversion results of the same response speech, the result is considered accurate. The text selected by majority vote is considered a highly reliable result and is subsequently reflected in the dementia test process. The final text conversion result selected by majority vote is used as part of the dementia test process, and the converted text serves as the basis for the test scoring as the output of the STT module 370. The STT module 370 analyzes the content of the user's voice responses through accurate text conversion, evaluates each voice response, and scores it. This scored result serves as a basis for judgment in the dementia test process and serves as an important factor in assessing the user's cognitive ability, verbal memory, and expressive ability.

FIG. 2 is an exemplary diagram illustrating a dementia test process performed by a dementia test device according to one embodiment of the present disclosure.

As illustrated in FIG. 2, the dementia test according to one embodiment of the present disclosure may be performed by performing a question-and-answer session for dementia test on at least one of the dementia patient terminal 100 or the caregiver terminal 200 connected to an AI call, and converting and analyzing the voice responses obtained from at least one of the dementia patient terminal 100 or the caregiver terminal 200 into text data.

First, the dementia test device 100 (refer to FIG. 1) that receives an inbound request including the first data including a dementia test request from at least one of the dementia patient terminal 100 or the caregiver terminal 200 sends an artificial intelligence (AI) call to at least one of the dementia patient terminal 100 or the caregiver terminal 200 of the test subject according to the inbound request. The computing device 300 performs a dementia test based on voice question and answer to at least one of the dementia patient terminal 100 or the caregiver terminal 200 of the user to which the AI call is connected, and the response voice of the test subject during the test may be acquired through at least one of the dementia patient terminal 100 or the caregiver terminal 200 and transmitted to the computing device 300.

The computing device 300 may convert the test subject's response voice received from at least one of the dementia patient terminal 100 or the caregiver terminal 200 of the user into text data by the STT module 370. Meanwhile, the test subject's response voice may include not only words intended to answer questions provided during the dementia test, but also words not intended to answer questions, such as interjections and monologues. For example, referring to the text data into which the response voice is converted as shown in FIG. 2, the response voice may include “puppy,” “cat,” “what is there?”, “horse,” and the like. Here, “puppy,” “cat,” and “horse” correspond to words intended to answer questions provided during the dementia test, and thus may be meaningfully utilized in analyzing the test content. On the other hand, “what is there?” is a monologue not intended to answer questions, and thus is not helpful in analyzing the test content. Words that are part of self-talk are treated as noise and are not included in the dementia test score calculation process. The computing device 300 extracts and analyzes only words that may be meaningfully utilized in the test content analysis from the text data, thereby improving the efficiency and accuracy of the test.

Based on the extracted words, the computing device 300 calculates a numerical value that may determine the presence or absence of dementia or the level of dementia symptoms in the test subject. By comparing and analyzing the numerical value with a pre-stored database for each test group, the computing device 300 may determine whether the test subject needs to visit a testing institution for follow-up testing.

FIG. 3 is a conceptual diagram illustrating a portion of an inbound request of a dementia testing device according to one embodiment of the present disclosure.

The dementia testing process of the computing device 300 of the inbound type is initiated by the first data including a dementia testing request. The first data may be generated by various inbound requests 300. For example, data may be collected through one or more of the following methods: making a phone call, scanning a two-dimensional barcode, entering a web address, sending an SMS message, touching an app push, touching an NFC tag, or entering a kiosk. The first data is typically generated by at least one of the dementia patient terminal 100 or the caregiver terminal 200 such as a smartphone owned by a user wishing to participate in the dementia test. However, the first data may also be generated by a desktop PC, a kiosk, or a computer with an input device connected to the terminal, and transmitted to the computing device 300. At least one of the dementia patient terminal 100 or the caregiver terminal 200 may be a mobile device owned by the test subject, or may be a tablet PC, desktop PC, kiosk, or a computer with an input device connected to the terminal.

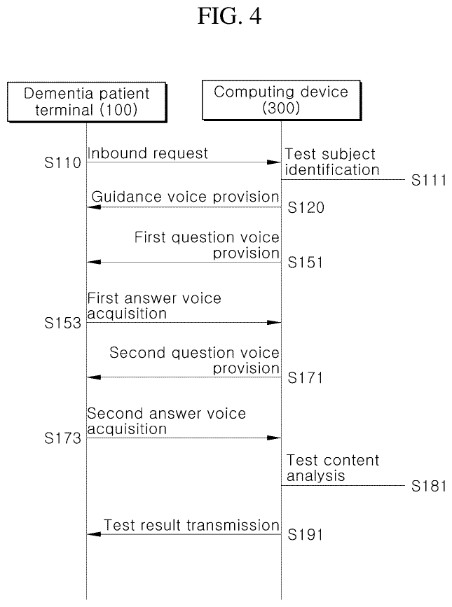

Since the first data is generated by a user possessing at least one of the dementia patient terminal 100 or the caregiver terminal 200, failure to properly control this process may result in excessive dementia test processes, potentially causing server downtime or network paralysis. Excessive inbound requests may lead to budget waste and significantly impact the progress of dementia management projects. Therefore, a test subject verification step (step S111 in FIG. 4) is essential, and the dementia testing device of the inbound type needs to handle “dementia test requests” with precision. To prevent repetitive dementia test request signals, the first data needs to be generated and analyzed for information about at least one of the dementia patient terminal 100 or the caregiver terminal 200, and a determination needs to be made as to whether the inbound request 300 is from the same user.

Since the inbound request 300 may be transmitted to the computing device 300 through various methods, such as making a phone call, scanning a two-dimensional barcode, entering a web address, sending an SMS message, tapping an app push, touching an NFC tag, or entering a kiosk, a determination needs to be made as to whether the request is from the same user. To determine this, the second data and consent to the use of personal information may be required. The inbound request 300 is generated by at least one of the dementia patient terminal 100 or the caregiver terminal 200. Since at least one of the dementia patient terminal 100 or the caregiver terminal 200 is a communication-connected device such as a smartphone, computer, kiosk, or tablet PC, reference information may be obtained to analyze a unique protocol for communication and determine whether the user is the same based on a unique address such as a MAC address.

FIG. 4 is a flowchart schematically illustrating a portion of an inbound request-based dementia test method according to an embodiment of the present disclosure.

As illustrated in FIG. 4, a dementia testing method according to an embodiment of the present disclosure may include an inbound request step (step S310), a test subject identification step (step S111), a guidance voice provision step (step S320), a first test execution step (steps S151 and S153), a second test execution step (steps S171 and S173), a test content analysis step (step S181), and a test result transmission step (step S191).

The inbound request step (step S310) is a step of collecting the first data including a user's dementia test request. The dementia testing process of the computing device 300 of the inbound type is initiated by the first data including the dementia test request. The first data may be collected through one or more of a phone call, a two-dimensional barcode capture, a web address input, an SMS text message transmission, an app push touch, an NFC tag contact, and a kiosk input. The first data is mostly generated by at least one of the dementia patient terminal 100 or the caregiver terminal 200, such as a smartphone, owned by a user who wishes to participate in a dementia test. However, it may also be generated by a desktop PC, a kiosk, a computer with an input device connected, and the like, other than a smartphone, and transmitted to the computing device 300. At least one of the dementia patient terminal 100 or the caregiver terminal 200 may be a mobile device owned by the test subject, and may be a tablet PC, a desktop PC, a kiosk, or a computer with an input device connected. From the perspective of the computing device 300, a “dementia test request” is received first, so it is called an “inbound request.”

The test subject identification step (step S111) is a step to prevent repeated dementia test requests from the same user. The same user may be using the present disclosure. While it is possible to determine whether dementia is improving by obtaining dementia test results periodically using the computing device 300 according to one embodiment of the present disclosure, due to budget constraints for dementia testing conducted by dementia management organizations and medical institutions, repeated testing on the same individual must be prevented in advance. Furthermore, there is a need to conduct dementia testing on potential dementia patients above a certain age range in each administrative district. Therefore, the test subject identification step (step S111) allows only users above a certain age range to proceed with the dementia test process.

The guidance voice provision step (step S320) is a step in which the computing device 300 provides voice guidance to a user who is a subject of a dementia test through at least one of the dementia patient terminal 100 or the caregiver terminal 200. In one embodiment, the voice guidance may include information about the subject (recipient) of the test, information about the test institution, information about the test solution (program), and information about rewards provided upon completion of the test. At this time, the subject (recipient) information is identified based on the first and second data obtained in the inbound request step. This information is inserted into the guidance voice, allowing for customized guidance voice to be provided to each subject. This may improve the participation and completion rates of the subjects receiving the AI call. After the guidance voice is provided, the dementia test may be conducted, which may include the first test and the second test, which are performed sequentially.

The first test execution step (steps S151 and S153) is a step where the first test, which is a practice test, is conducted and the subject (at least one recipient of the terminal dementia patient 100 and the terminal caregiver 200) understanding of the test progress is improved. The first test execution step may include a step (step S151) in which the computing device 300 provides a first question voice to at least one of the dementia patient terminal 100 or the caregiver terminal 200, and a step (step S153) in which the computing device 300 obtains a first answer voice from at least one of the dementia patient terminal 100 or the caregiver terminal 200.

Here, the first question voice may include a voice requesting an answer to the first topic during the first time period, and the first answer voice may include a voice in which the test subject answers in response to the first question voice.

The test subject may participate in the first test by responding to the first question voice. The first answer voice of the test subject may be acquired through a sound acquisition unit of at least one of the dementia patient terminal 100 or the caregiver terminal 200, and transmitted to the computing device 300 through a communication unit of at least one of the dementia patient terminal 100 or the caregiver terminal 200.

In one embodiment, the first answer voice acquired through the first test may be used as data to determine whether the subject properly understood the test procedure. That is, the dementia test method based on AI-call according to one embodiment of the present disclosure may further include a step of determining the understanding level of the test procedure of the test subject based on the first answer voice.

For example, the computing device 300 may calculate a value of the understanding level of the test procedure of the test subject and determine whether to proceed with the test based on the value. Here, the “value of the understanding level” is a numerical value indicating the degree of understanding of the test subject's test procedure. Specifically, the computing device 300 calculates the value of the understanding level based on the ratio of the words spoken in response to the first question voice among the first answer voices and the other voices. In the case that the calculated value of the understanding level is below a preset value, the second test may not be conducted.

In another specific example, the computing device 300 calculates the value of the understanding level based on the number of words spoken in response to the first topic presented in the first test. In the case that the calculated value of the understanding level is below a preset value, the second test may not be conducted. In the case that the test subject is determined to not properly understand the test procedure and the second test is not conducted, the computing device 300 may provide a voice guide to guide the test procedure or schedule a later test, but is not limited thereto.

In this case, the computing device 300 may provide a voice guiding the examination process or may schedule a subsequent examination, but is not limited thereto.

In another embodiment, the step of acquiring a first response voice from at least one of the dementia patient terminal 100 or the caregiver terminal 200 in the first examination process may be omitted. Since the first examination is merely a test to enhance the understanding of the examination process and is not used as data to determine the language fluency of the examinee, acquisition of the response voice for the first examination may be omitted. This reduces the total amount of data transmitted and received during the examination process.

The second test execution step (steps S171 and 473) is a step for obtaining voice data used to determine the presence or absence of dementia and the level of dementia symptoms while conducting the second examination, which is the main examination.

Referring to FIG. 4, the second test execution step may include a step (step S171) in which the computing device 300 provides a second question voice to at least one of the dementia patient terminal 100 or the caregiver terminal 200, and a step (step S173) in which the computing device 300 obtains a second answer voice from at least one of the dementia patient terminal 100 or the caregiver terminal 200.

Here, the second question voice may include a voice requesting an answer to a second topic during a second time, and the second answer voice may include a voice in which the test subject answers in response to the second question voice.

The test subject may participate in the second test by responding to the second question voice, and the second answer voice of the test subject may be acquired through the sound acquisition unit of at least one of the dementia patient terminal 100 or the caregiver terminal 200, and may be transmitted to the computing device 300 through the communication unit of at least one of the dementia patient terminal 100 or the caregiver terminal 200.

The test content analysis step (step S181) is a step in which the computing device 300 analyzes the test content based on the second answer voice acquired from at least one of the dementia patient terminal 100 or the caregiver terminal 200.

In the test content analysis step (step S181), the dementia test module 320 (refer to FIG. 1) of the computing device 300 may analyze the test content by converting the second response voice received from at least one of the dementia patient terminal 100 or the caregiver terminal 200 into text data and calculating a language fluency value based on the converted text data. Specifically, the test content analysis step (step S181) may include a step of converting a second response voice received from at least one of the dementia patient terminal 100 or the caregiver terminal 200 into text data, a step of extracting at least one word corresponding to a second topic of the second test from the converted text data, a step of calculating a language fluency value based on at least one word corresponding to the extracted second topic, a step of comparing and analyzing the language fluency value with a preset reference value, and a step of determining a subject for a follow-up test in the case that the language fluency value is lower than the preset reference value, and a step of determining a subject not to be tested in the case that the language fluency value is greater than or equal to the preset reference value.

The step of calculating the language fluency value may include a step of scoring by applying an addition/deduction point standard to at least one of the total number of words, the number of words in the first half, the number of words in the second half, the number of characters per word, the number of category changes, the number of words per category, or the number of duplicate words. Additionally, the preset reference values for comparing the calculated language fluency values may be differentially set for each test group classified based on auxiliary information including at least one of gender, age, education level, and number of cohabitants. The calculated language fluency value may be compared and analyzed with the reference value set for the group to which the test subject belongs.

Meanwhile, in one embodiment, the test content analysis step (step S181) may further include a step of analyzing changes in the test subject's speaking style based on the previous test data of the test subject and the second response voice acquired through the current test, in the case that the test subject has a test history, that is, in the case that at least one of the dementia patient terminal 100 or the caregiver terminal 200 connected to the AI call has a test history. Here, “speaking style” may include, but is not limited to, speaking speed and pronunciation accuracy.

The dementia test module 320 (refer to FIG. 1) of the computing device 300 may analyze the speech style of the second answer voice obtained in this test and compare it with the speech style analysis data of the answer voice obtained in the existing test to calculate the speech style change value. The dementia test module 320 of the computing device 300 may analyze the speech style in the case that the change value exceeds a specific value (a preset value), the subject may be determined as a follow-up test subject. Furthermore, the dementia test module 320 of the computing device 300 may determine a subject as a follow-up test subject in the case that the difference between the language fluency value calculated in the previous test and the language fluency value calculated in the current test exceeds a specific value (a preset value). This allows for the prevention of rapid worsening of symptoms by performing follow-up testing not only in cases where the absolute language fluency value is high but also in cases where language fluency has relatively decreased compared to the previous test.

In one embodiment, the test content analysis step (step S181) may further include a step of determining the test subject's understanding of the test procedure based on the first response voice. Specifically, the computing device 300 may analyze the first response voice to determine whether the subject fully understands the test procedure and is responding, or whether the subject is responding without understanding and is responding unrelated to the test content. For example, in the case that the subject answers a preset number of words about the first topic presented in the first test, the computing device 300 may determine that the subject properly understands the test procedure, and otherwise, determine that the subject does not properly understand the test procedure.

Even in the case that the language fluency value calculated by analyzing the second response voice is below a preset reference value, in the case that the comprehension value of the test procedure calculated by analyzing the first response voice is below a specific value (preset value), the computing device 300 may not immediately determine the subject as a subsequent test subject but may conduct a retest after re-introducing the test procedure. This makes it possible to distinguish cases where a subject with excellent language fluency (without dementia symptoms) receives a low language fluency value due to a lack of understanding of the test procedure, thereby improving the accuracy of the test.

The test result transmission step (step S191) is a step for transmitting the test result analyzed in the test content analysis step (step S181) to at least one of the dementia patient terminal 100 or the caregiver terminal 200.

In one embodiment, the test result may be transmitted in the form of a message, and the message may include a link for viewing the test result. The method for transmitting the test result is not limited to the examples described above and may be provided in various forms, such as by phone, mail, or message. Furthermore, the recipient of the test result is not limited to the test subject, but may also include a variety of people around the test subject, such as a cohabitant or caregiver.

In one embodiment, the test result may include information on whether the test subject qualifies for follow-up testing and information on predicting future symptoms. Here, the symptom prediction information is derived based on symptom information from a similar group of individuals with similar auxiliary information (e.g., age, gender, education level, number of cohabitants, etc.) to the test subject. This information may include trends in language fluency decline over time, follow-up test results, and the like.

FIG. 5 is an exemplary diagram illustrating a STT multiplexing process in a dementia test device according to an embodiment of the present disclosure.

The processor 330 may control the STT module 370 including one or more speech-to-text (step STT) models, convert the response voice into text by applying three or more speech-to-text models, compare the words in the converted text, and selects the converted text that accounts for the majority of the converted text to score the results of the dementia test process. This serves as a voting system for assessing the accuracy of the speech-to-text conversion process. Because elderly dementia test participants may have slurred speech, accurate voice analysis using existing STT engines may be difficult. Therefore, it is desirable to use three or more STT engines to convert speech to text using the closest word. For example, in the case that a dementia test participant pronounces “tiger”, a specific STT engine may convert it to “deer”. However, in the case that the other two STT engines convert it to “tiger,” the word “tiger” may be selected at a 1:2 ratio to proceed with the dementia test. This process improves the accuracy of the dementia test.

Below, a system, device, and method for improving cognition of a dementia patient are described, based on the result of a dementia test performed using at least one of FIGS. 1 to 5. In particular, the present disclosure is to improve the cognition of a dementia patient by providing at least one appropriate mission based on the dementia status of the patient as determined by the dementia test result. Various embodiments of this method are disclosed below.

First, in relation to the present disclosure, there are various types of dementia, and each type of dementia has different causes and symptoms. For example, Alzheimer's dementia is the most common form of dementia and is characterized by memory loss and gradual deterioration of cognitive function. Vascular dementia is caused by cerebrovascular disease and may occur after a stroke. Lewy body dementia is caused by the accumulation of abnormal protein clumps called Lewy bodies in the brain, and is characterized by hallucinations, movement disorders, and cognitive decline. Frontotemporal dementia is caused by damage to nerve cells in the frontal and temporal lobes, and is characterized by personality changes and decreased language ability. Alcoholic dementia is caused by long-term excessive drinking and is characterized by memory loss and cognitive decline. Other types of dementia include Parkinson's disease dementia, Creutzfeldt-Jakob disease, and hydrocephalus. Since the causes and symptoms of each type of dementia are different, the present disclosure provides a mission appropriate for each type of dementia, thereby aiming to improve the cognition of the target dementia patient.

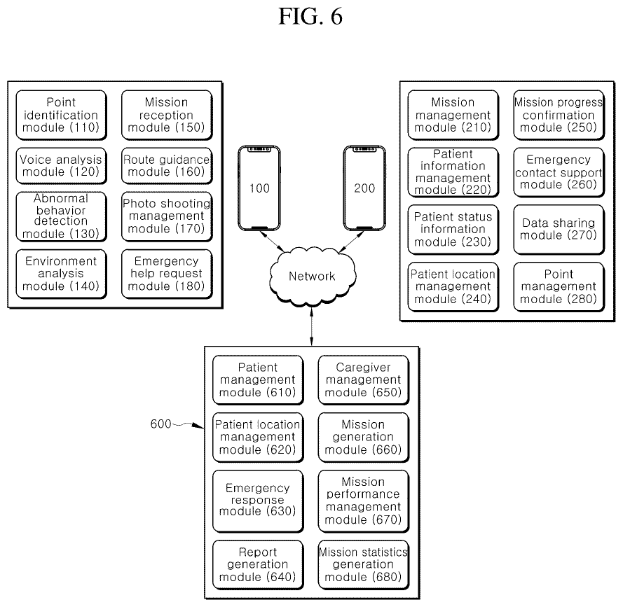

FIG. 6 illustrates a dementia patient cognitive improvement system according to an embodiment of the present disclosure.

Referring to FIG. 6, a dementia patient cognitive improvement system may include a first terminal 100, a second terminal 200, and a mission processing device 600. In this case, the first terminal 100 may represent the terminal of a target dementia patient, and the second terminal 200 may represent a terminal of a target caregiver of the target dementia patient. Meanwhile, the mission processing device 600 may be the computing device (e.g., 300 of FIG. 1) or a component thereof. However, for convenience of description, the mission processing device 600 will be described below as the computing device 600.

Referring to FIG. 6, the first terminal 100, the second terminal 200, and the computing device 600 may be interconnected to perform various operations, such as processing, monitoring, and providing feedback on the mission performance of a target dementia patient.

As described above, the present disclosure is to improve the cognition of a target dementia patient through appropriate missions based on their dementia status. Here, the dementia status is determined based on at least one of the dementia patient's attributes, the degree (or level, severity, etc.) of dementia, and the type of dementia. Meanwhile, when determining the dementia status, the performance or function (e.g., sensors) of the terminal possessed by the dementia patient may be further referenced to determine whether the mission has been performed.

In the present disclosure, the computing device 600 provides a mission to the first terminal 100, and a comprehensive assessment is made of the target dementia patient's participation in the mission, whether the mission is successfully completed, and appropriate points are awarded accordingly. These points may be related to the difficulty of the next mission, or may be used as capital for the dementia patient's mission, or as cash assets for personal needs outside of the mission. The nature or type of these points may be determined by the dementia patient or their caregiver's choice.

The computing device 600 according to the present disclosure may, for example, generate and provide customized missions based on the current location (e.g., residence) of the dementia patient and/or their caregiver.

As described above, a selection of the generated mission or request for a modification to the mission may be performed by a caregiver. In the case that the caregiver requests a modification to the mission, the computing device 600 records the request, determines whether to modify the mission, and determines the extent of the modification. The computing device 600 may then modify and re-provide the mission accordingly.

Alternatively, in the case that the caregiver requests a modification to the mission selected by the caregiver, the computing device 600 may calculate the difficulty of the mission and suggest the time required to perform the mission and the points provided upon completion of the mission. The caregiver may also re-modify the time and points according to the suggestion and suggest them.

In the present disclosure, a caregiver may refer to a legal caregiver with a kinship relationship or a person with a professional license and recognized by a public institution. It is preferable that the computing device 600 perform a sufficient qualification review and authentication procedure in advance for the caregiver before registration. In addition, the computing device 600 may expose at least some of the caregiver's personal information in various ways to enable a third-party review of the qualifications. Meanwhile, in the case that the latter caregiver is qualified, the computing device 600 may be registered to act as a caregiver for multiple dementia patients.

According to the present disclosure, through the first terminal 100, various other IoT devices, or CCTV, the computing device 600 may monitor the target dementia patient in real time, and in the case that an emergency situation occurs for the target dementia patient while performing a mission, the emergency situation may be notified to not only the second terminal 200, but also to a dementia care center, a medical institution, and the like, and support may be requested.

Referring to FIG. 6, the dementia patient cognitive improvement system according to the present disclosure may be structured such that a mission is generated by the computing device 600 and transmitted to the second terminal 200, which then transmits the mission to the first terminal 100. This is because even in the case that the mission is directly transmitted to the target dementia patient, it is difficult to be certain whether the mission will be recognized, selected, modified, or otherwise acted upon. Furthermore, in the case of dementia patients, it is difficult to directly determine whether the patient's health or psychological state changes at any given moment, or what situation the patient is in, such as whether the patient is currently capable of performing the mission. Therefore, it is preferable to transmit the mission through a caregiver or to simultaneously transmit it to the target dementia patient and the caregiver.

In the present disclosure, the mission may be performed using the display, microphone, speaker, camera, GPS, accelerometer, gyroscope, and the like of the first terminal 100, and may include various levels of missions that may induce cognitive improvement in dementia patients in their daily lives.

In this regard, the computing device 600 may obtain a mission template created by an expert, such as at a dementia care center, and provide it to the second terminal 200 for selection by the caregiver.

The computing device 600 may obtain, record, or store mission performance and status information. In this regard, the first terminal 100 may obtain mission performance status data of the target dementia patient according to mission performance (e.g., GPS, photography, etc.) and transmit and share this data with the second terminal 200, a mission management device (e.g., a dementia care center or medical institution), and the like. During the sharing, the computing device 600 may provide an interface, such as a chat window or video conference, to enable data communication between at least the second terminal 200 and the mission management device according to the mission execution.

The computing device 600 may monitor various actions related to the target dementia patient's mission execution in real time and perform emergency response measures. At this time, the computing device 600 may directly or indirectly monitor the patient's condition through the second terminal 200 and the dementia care center, and may request emergency assistance in the event of an abnormal situation.

When the target dementia patient's mission is completed, the computing device 600 may analyze the performance of the mission execution and provide feedback based on the analysis results. For example, the computing device 600 may analyze the mission execution result data, provide a report to the caregiver and the dementia care center, and recommend the next customized mission for the target dementia patient in advance. In the case that the patient successfully completes a mission, for example, within a given time limit, the computing device 600 may recommend a more difficult mission to the caregiver than the previously provided mission. The computing device 600 may also provide points based on the success or failure of the mission, thereby motivating not only the success of the mission but also the performance of the mission itself.

Next, each component of the dementia patient cognitive improvement system of FIG. 6 will be described in more detail.

First, the configuration of the dementia patient terminal, that is, the first terminal 100, which performs a mission for cognitive improvement and supports monitoring of the dementia patient's condition, will be described.

The first terminal 100 may include at least one of a point identification module 110, a voice analysis module 120, an abnormal behavior detection module 130, an environment analysis module 140, a mission reception module 150, a route guidance module 160, a photo shooting management module 170, and an emergency help request module 180. Some of the above modules may be integrated and implemented as a single component. In addition, some of the functions of the above modules may be implemented in the form of an application.

The point identification module 110 is configured to allow the target dementia patient to check the points accumulated when the target dementia patient completes a mission. These points are intended to encourage a sense of accomplishment and motivation to complete the mission in accordance with the target dementia patient's performance.

The voice analysis module 120 analyzes the dementia patient's voice commands and responses to provide appropriate feedback. This allows the patient's cognitive status to be identified through voice data analysis.

The abnormal behavior detection module 130 detects unexpected behaviors (e.g., leaving a specific area, prolonged inactivity) of the dementia patient. This module recognizes the patient's unusual behavior and promptly notifies the caregiver, dementia care center, or the computing device 600.

The environment analysis module 140 detects and analyzes the environment (e.g., noise, temperature, location, etc.) of the dementia patient's location. This module determines whether the environment is conducive to safe mission execution, thereby ensuring safety.

The mission reception module 150 receives missions set by the caregiver and provides them to the patient. This module confirms the mission details and provides guidance on how to proceed. At this time, the guidance may be generated and provided through at least one, or a combination of, visual, auditory, or tactile elements, depending on the dementia patient's dementia status.

The route guidance module 160 is configured to provide a route to the mission execution location based on GPS, for example, and is intended to support the dementia patient's safe arrival from the starting point to the destination through various methods, such as visual and audio. The provided route may be an optimal route. Meanwhile, the optimal route is determined based on the dementia patient's dementia status and does not simply mean the shortest route. Safety may be prioritized during the dementia patient's mission execution. However, the optimal route may be modified at the caregiver's discretion.

The photo shooting management module 170 is configured to manage the photography and video recording of the dementia patient during the mission and transmit the results to the caregiver. It is intended to confirm the dementia patient's mission completion and provide visual data to the caregiver, dementia care center, or the computing device 600.

The emergency help request module 180 is configured to request help from a caregiver and a dementia care center in the event of an emergency for a dementia patient. It is intended to immediately transmit a notification signal via a button or voice command (e.g., an emergency request voice). In addition, the emergency help request module 180 may output the current dementia patient's status and information in a speakerphone format, thereby requesting help from those around them first or simultaneously with the notification signal.

Next, the configuration of the caregiver terminal, that is, the second terminal 200, which monitors the status of the target dementia patient and performs a mission management function, will be described.

This second terminal 200 may include at least one of a mission management module 210, a patient information management module 220, a patient status information module 230, a patient location management module 240, a mission progress confirmation module 250, an emergency contact support module 260, a data sharing module 270, or a point management module 280.

The mission management module 210 is configured to allow a caregiver to create, modify, and delete missions suitable for a dementia patient. It may also receive missions provided by a dementia care center or the computing device 600 and modify them to suit the first terminal 100. It may also set customized missions that suit the patient's condition, dementia degree, and the region and environment of the caregiver and dementia patient.

The patient information management module 220 is configured to manage the dementia patient's personal information, dementia diagnosis results, and the like, and the mission recommended by the mission management module 210 may vary depending on the dementia diagnosis results. As described above, the dementia diagnosis results are classified into CDR grades (e.g., 0, 0.5, 1, 2, 3, 4, and 5) that indicate the degree, level, or severity of the dementia patient, and missions are provided only to patients corresponding to 0 to 2, and no missions are provided to patients corresponding to 3, 4, and 5, or the lowest level of missions may be uniformly and repeatedly provided until a certain point in time. The patient information management module 220 is used as basic data for mission setting and monitoring, and may check whether the dementia patient's cognitive level has improved, and accordingly, may check the frequency of mission performance, the degree of cognitive improvement compared to the mission, and the like.

The patient status information module 230 is configured to monitor the real-time status (e.g., location, activity level, etc.) of a dementia patient, and may transmit a notification signal when the dementia patient's safety is confirmed or abnormal behavior is detected.

The patient location management module 240 is configured to enable a caregiver to check the patient's current location based on location information such as the dementia patient's GPS. It may be very important to ensure that dementia patients do not deviate from the provided or safe path while performing a mission. For example, if a dementia patient leaves the house to perform a mission, confirming the patient's location may be crucial.

The mission progress confirmation module 250 is configured to check whether the dementia patient has performed the mission, the degree of mission completion, and whether the mission has been completed. It may monitor the dementia patient's mission performance status and provide feedback based on the monitoring results.

The emergency contact support module 260 is configured to provide the ability to immediately contact a dementia care center, medical institution, or the computing device 600 when a dementia patient requires assistance. This allows for not only a simple voice call but also the transmission of information regarding the dementia patient's location and situation. The caregiver may receive information about the dementia patient's current status and situation and provide relevant information to the dementia care center, medical institution, etc. via the computing device 600, enabling prompt follow-up.

The data sharing module 270 is configured to enable real-time sharing of dementia patient status data with dementia care centers, medical institutions, and the like, via the computing device 600. Data may be processed to share communication between dementia patients and their caregivers, dementia patient status information, and situational information with dementia care centers, medical institutions, and the like, via the computing device 600. This allows information to be provided to dementia care centers, medical institutions, and the like, enabling collaboration to manage dementia patient status and analyze mission performance.

The point management module 280 is configured to manage points earned by dementia patients or set rewards for each mission. These points are intended to motivate dementia patients to complete their missions.

Finally, the mission management device, that is, the computing device 600, which functions as a server or system to support dementia patients and their caregivers and to integrate and manage data to support cognitive improvement in dementia patients, will be described.

The computing device 600 may include at least one of a patient management module 610, a patient location management module 620, an emergency response module 630, a report generation module 640, a caregiver management module 650, a mission generation module 660, a mission performance management module 670, and a mission statistics generation module 680.

The patient management module 610 is configured to comprehensively manage the dementia patient's status, diagnosis information, mission performance data, and the like, and may track the dementia patient's dementia progression status and analyze data related thereto.

The caregiver management module 650 manages caregiver information and allows users to check matching information with dementia patients (e.g., enabling multiple patient management), the number of missions assigned to each dementia patient, and whether each mission is provided to each dementia patient. This module aims to facilitate smoother communication and support for the dementia patient.

The patient location management module 620 monitors and records the dementia patient's location data in real time. In the case that the dementia patient deviates from a pre-designated route, the module immediately notifies the caregiver and adjusts the management monitoring level to enhance support.

The emergency response module 630 transmits a notification signal and provides response guidelines to the caregiver or other dementia care centers or medical institutions near the dementia patient in the event of an emergency. This module prioritizes the safety of the dementia patient.

The report generation module 640 generates an activity report based on the dementia patient's mission performance data. This allows the caregiver to determine the missions provided to the dementia patient and the mission success rate. It also generates reports on the extent of the dementia patient's cognitive improvement, including information on the frequency of the caregiver's mission provision. This allows the patient, caregiver, dementia care center, or medical institution to receive information on the patient's cognitive improvement performance through missions.

The mission generation module 660 automatically generates appropriate missions based on the dementia patient's dementia status, patient attributes, and caregiver's circumstances. It may recommend missions to maximize the effectiveness of dementia cognition improvement. The patient's attributes (or environment) may include at least one of the following: age, gender, education level, residence, hometown, religion, presence of a spouse (or caregiver), income level (e.g., fixed income such as a pension), general health (presence of other chronic diseases), allergies, surrounding public safety, walking difficulty, and use of a walking aid. However, this is not limited thereto.

The mission performance management module 670 tracks the mission progress of a dementia patient in real time and may provide mission success records and feedback.

The mission statistics generation module 680 analyzes the patient's activity performance and improvement effects based on accumulated data regarding mission performance, thereby providing a data-based plan for cognitive improvement of the target dementia patient.

Each of the missions may or may not have a priority, and each may be assigned a weight, which may vary depending on the degree of the weight. This may also be linked to the provided points.

As described above, the computing device 600 may generate and provide missions based on the dementia status.

For example, when generating a mission, the computing device 600 may consider the attributes of the dementia patient, as described below.

For example, age is an attribute, and it is preferable to consider differences in the physical and cognitive abilities of dementia patients based on age. For example, relatively simple, less physically demanding missions may be appropriate for older dementia patients. For example, a younger dementia patient (in their 60s) might be given an active mission, such as “walk 1 km and take pictures of the surroundings,” while an older dementia patient (in their 80s or older) might be given a simpler mission, such as “find a specific object in the house.”

For example, gender is an attribute that allows for setting missions that reflect gender-specific interests and activity preferences. For example, a female dementia patient might be given a life-centered mission, such as “visit a traditional market and take pictures of cooking ingredients,” while a male dementia patient might be given a hobby-based mission, such as “find sporting goods related to their hometown.”

For example, a higher level of education might be given missions that involve complex information processing and problem solving, while a lower level of education might be given simple, visually stimulating missions. For example, a highly educated dementia patient might be given a mission like “Take a history quiz and send a photo of the results,” while a less educated dementia patient might be given a mission like “Find a red object in your neighborhood and take a photo.”

The residence attribute is intended to provide missions that reflect the differences in living environments, such as urban and rural environments. For example, a dementia patient who lived in an urban area might be given a mission like “Take a photo of a receipt at a nearby cafe,” while a dementia patient who lived in a rural area might be given a mission like “Take photos of three types of vegetables in the garden.”

The hometown attribute, for example, is intended to encourage emotional stability through reminiscence by stimulating memories related to hometown. For example, a dementia patient might be given a mission like “Visit a store selling traditional hometown food and take a photo” or “Find a place reminiscent of hometown scenery (e.g., a mountain or river).”

The religious attribute, for example, aims to provide emotional stability and a sense of belonging to dementia patients through religious activities. For example, missions based on religion may be provided, such as “Attend a church service and take a photo,” for Christianity, “Visit a temple and take a landscape photo,” for Buddhism, or “Take a tree photo after a walk in a nearby park.”

The presence of a spouse (or caregiver) attribute, for example, provides additional support for completing missions if a caregiver is present. Therefore, missions that may be completed independently without a caregiver should be set. For example, a mission like “Visit a cafe with a caregiver and take a photo” could be provided for a dementia patient with a spouse, while a mission like “Walk a certain distance alone and take a landscape photo” could be provided for a dementia patient without a spouse.

The income level attribute, for example, is designed to consider the costs required to complete the mission. For example, for dementia patients with high income or sufficient assets, a mission such as “take a picture of the receipt after eating at a nearby restaurant” could be provided, while for patients with relatively low income or not sufficient assets, a mission such as “take a picture of the bench after a walk in the neighborhood park” could be provided.

General health (presence of other chronic diseases) as an attribute, for example, is to limit the intensity of physical activity or consider additional health-related missions of a dementia patient. For example, a dementia patient in good health might be given a mission like “take a picture of a tree after a 1 km walk.” A dementia patient with a chronic illness, for example, might be given a mission like “film a stretching video at home.”

The allergy attribute, for example, is intended to avoid missions involving allergens or provide alternative missions. For example, a dementia patient with allergies might be given a mission like “find an allergy-free ingredient and take a picture,” while a dementia patient without allergies might be given a mission like “buy fruit at a nearby supermarket and take a picture.”

The security level attribute, for example, is intended to design indoor-focused missions in areas with poor security, while providing outdoor-focused missions in areas with good security. For example, a dementia patient living in a less safe environment might be given a mission like “Organize family photos at home and take a photo.” Conversely, a dementia patient living in a relatively safe environment might be given a mission like “Take a 30-minute walk in a nearby park and take a photo.”

The walking difficulty attribute is intended to account for areas with many hills, high traffic volume, or stairs. High walking difficulty may be defined as, for example, walking to a destination with four or more crosswalks. Medium walking difficulty may be defined as, for example, walking to a destination with one to three crosswalks. Low walking difficulty may be defined as, for example, walking to a destination without a crosswalk. These walking difficulties may influence the creation of appropriate missions.

The use of a walking aid attribute is intended to limit the travel distance and activity type based on the use of a walking aid. For example, a dementia patient using an assistive device may be provided with a mission such as “Walk 100 meters and take pictures of the surrounding area,” while a dementia patient not using an assistive device may be provided with a mission such as “Walk more than 500 meters and take pictures of a park.”

As described above, the computing device 600 may generate and provide missions based on the dementia status.

For example, the computing device 600 may consider the dementia patient's CDR rating when generating a mission, as described below.

The patient's CDR rating may be considered when generating a mission. The CDR rating considered for mission generation may be combined with the attributes to influence the mission.

First, for CDR of 0 (normal cognitive function), a mission such as “Walk in a park and take pictures of the scenery” may be provided. To this end, the route guidance module 360 of the first terminal provides a movement route to the dementia patient, and after moving to the dementia patient's destination (e.g., a park), the patient may take a picture of the park scenery through the photo shooting management module 370 and press the mission completion button or take a picture of the scenery to immediately confirm the completion of the mission. Accordingly, the computing device 600 may provide appropriate points accordingly when the dementia patient walks along the designated route and sends a picture. Meanwhile, the second terminal may set the location of the park in the mission management module 210 and monitor the dementia patient's location in real time with the patient location management module 240. The computing device 600 may acquire the dementia patient's route and activity data in real time through the patient location management module 320, and when the patient's mission is completed, manage the relevant matters through the mission execution management module 370.

In case another mission is provided, for example, recording impressions after reading a book, the first terminal may check the mission content in the mission receiving module 350 and induce the dementia patient to record a simple impression (e.g., “It is fun”) using the voice analysis module 320 after reading the book. The computing device 600 may provide points accordingly when the dementia patient submits the impression through voice, and the like. Meanwhile, the second terminal may check whether the dementia patient has performed the mission through the mission management module 210. The computing device 600 may evaluate emotional stability based on the voice data analysis results and record the result in the patient management module 310, and when the mission is completed, record the result in the mission performance management module 370.

In the case that another mission is provided to take a picture after visiting a traditional market, the first terminal 100 may obtain a picture of the market using the photo shooting management module 370 after arriving at the traditional market through the route guidance module 360. The computing device 600 may provide points based on location information such as GPS when reaching a designated location and submitting a picture. The second terminal 200 may check the progress of the mission using the mission progress confirmation module 250, check the movement status of the patient using the patient location management module 240, and, if necessary, support the safe return of the dementia patient to the dementia care center, medical institution, or the computing device 600 using the emergency contact support module 260. The computing device 600 may monitor the location of the dementia patient through the patient location management module 320, and record in the mission performance management module 370 when the dementia patient completes the mission.

Another mission, in the case that taking a family photo after a video call is provided, the first terminal 100 may conduct a video call with a third party (e.g., family, relative, friend) designated by the second terminal 200, take a photo with the other party of the video call, and submit the photo through the photo shooting management module 370. The computing device 600 may provide points upon completion of the video call and submission of the photo. The second terminal 200 may select a mission from the mission management module 210 and assign the mission to the first terminal 100, and in the process of calling the contact information of the third party (e.g., family, relative, friend) from the dementia patient's terminal to make a video call. The computing device 600 may analyze activity data to update the patient's emotional stability and family relationship data.

Next, the mission for the case of CDR 0.5 (mild cognitive impairment) will be described.

First, when providing a mission such as visiting a pharmacy near home, the first terminal 100 may navigate to the pharmacy via the route guidance module 360 and take and transmit a photo of the pharmacy entrance via the photo shooting management module 370. The computing device 600 may confirm the pharmacy visit using GPS and a photo, and provide corresponding points upon submission of the photo. The second terminal 200 may monitor the patient's movement status in real time using the patient location management module 240. The computing device 600 may record pharmacy visit data in the patient location management module 320 to supplement health management data.

When providing another mission, such as finding an object of a specific color, the first terminal 100 may confirm the mission via the mission reception module 350 and submit an object of a specified color (e.g., red) to the photo shooting management module 370. The computing device 600 may provide corresponding points when a photo that satisfies the color conditions is submitted. The second terminal 200 may process the completion after verifying the photo through the mission progress confirmation module 250. The computing device 600 may record and analyze color recognition ability data through the patient management module 310. In another mission, when a mission such as finding a park bench is provided, the first terminal 100 may find a specific bench in the park through the route guidance module 360 and transmit it to the photo shooting management module 370. The computing device 600 may provide corresponding points when reaching the bench is confirmed through GPS and a photo. The second terminal 200 may monitor the location of the dementia patient through the patient location management module 240. The computing device 600 may evaluate spatial cognitive ability based on movement path data, accumulate points and determine whether the patient's cognitive ability has improved through the patient management module 310, and record mission performance data using the mission performance management module 370.

Next, the provision of missions in the case of CDR 1 (mild dementia) will be described.

First, when a mission such as finding an item at home is provided, the first terminal 100 may confirm the mission using the point identification module 310 and submit a specific item (e.g., a book) to the photo shooting management module 370. The computing device 600 may award points upon submitting a photo of the item. The second terminal 200 may confirm the submitted photo using the mission progress confirmation module 250. The computing device 600 may determine whether points have accumulated and the patient's cognitive ability has improved through the patient management module 310, and may record mission performance data with the mission performance management module 370.

In another mission, in the case that a mission such as taking a picture with a caregiver is provided, the first terminal 100 may take a picture with the caregiver and submit it to the picture shooting management module 370. The computing device 600 may provide points upon submission of the picture. The second terminal 200 may perform the mission together with the caregiver and transmit the result to the data sharing module 270. The computing device 600 may determine whether points have accumulated and the patient's cognitive ability has improved through the patient management module 310, and may record mission performance data with the mission performance management module 370.

In another mission, in the case that a mission such as taking a short walk is provided, the first terminal 100 may take a landscape photo after walking a short distance (e.g., 300 m) along the route guidance module 360. The computing device 600 may provide GPS data and corresponding points upon submission of the photo. The second terminal 200 may monitor the dementia patient's walking route in real time. The computing device 600 may record the dementia patient's movement data in the patient location management module 320.

In the case that another mission, such as taking a photo after tidying up, is provided, the first terminal 100 may tidy up a designated location at home (e.g., a desk, dining table, etc.) and then transmit the photo via the photo shooting management module 370. The computing device 600 may provide corresponding points upon submission of the photo of the tidy up state. The second terminal 200 may provide photo verification and feedback data via the mission progress confirmation module 250. The computing device 600 may analyze the patient's space utilization ability by recording organizing activity data in the mission execution management module 370.

Next, an example of mission provision in the case of CDR 2 (moderate dementia) will be described.

First, in the case that a mission, such as taking a picture of a specific object in the home, is provided, the first terminal 100 may confirm the mission in the point confirmation module 310 and transmit an object (e.g., a plate) designated by the caregiver through the photo shooting management module 370. The computing device 600 may provide points upon submitting a picture of the designated object. The second terminal 200 may perform mission creation and verification in the mission management module 210. The computing device 600 may store object recognition data in the mission execution management module 370 to track cognitive improvement.

For another mission, in the case that a simple activity with a caregiver is provided, the first terminal 100 may perform the simple activity (e.g., solving a puzzle) with the caregiver and send a photo upon completion. The computing device 600 may provide corresponding points when the photo of the activity is sent. The second terminal 200 may transmit completion data via the activity support and data sharing module 270. The computing device 600 may analyze emotional exchange data to assess the level of interaction between the patient and the caregiver.

For another mission, in the case that a short walk is provided, the first terminal 100 may take a walk in a park near the home with the caregiver via the route guidance module 360 and send a photo via the photo shooting management module 370. The computing device 600 may provide corresponding points when the patient moves along the designated route and submits the photo. The second terminal 200 may monitor the dementia patient's movements in real time and ensure their safety. The computing device 600 may record and analyze the patient's physical activity data based on the patient's movement path data.

In the case that, for another mission, a mission such as selecting a favorite food, the first terminal 100 may, for example, view at least two food images in an application and select the preferred food using the voice analysis module 320. The computing device 600 may provide corresponding points upon data submission after selection. The second terminal 200 may provide the patient's preference data to a dementia care center or medical institution via the computing device 600 via the data sharing module 270 and provide feedback. The computing device 600 may analyze the food selection data to evaluate and record emotional responses and preferences.

Each of the aforementioned missions may or may not have a priority, and each may be assigned a different weight, depending on the degree of the weight. This may also be linked to the provided points.

As described above, the computing device 600 may generate and provide missions based on the dementia status.

For example, the computing device 600 may consider the type of dementia patient when generating a mission, as described below.

The computing device 600 may consider the type of dementia when generating a mission, and this may be combined with at least one of the dementia attributes and dementia degree CDR information.

When the computing device 600 designs missions that consider the patient's cognitive and behavioral characteristics for each dementia type, it may more effectively promote cognitive improvement and an enhanced quality of life.

First, in cases of Alzheimer's dementia, memory decline and loss of orientation to time and place are frequent. Accordingly, the computing device 600 may provide at least one of the following customized missions: a memory stimulation mission, for example, speaking out names and relationships after viewing a family photo; a route learning mission, for example, taking a photo after visiting a dementia care center following GPS guidance; a daily recollection mission, for example, taking a photo of a place visited in the past (e.g., a symbolic place in the hometown) and submitting it; a schedule management mission, for example, checking a schedule displayed on a display and performing a mission (e.g., pressing a notification button after taking medication).

Next, in cases of vascular dementia, language impairment, mood swings, and difficulty with physical activity are common. Therefore, the computing device 600 may provide at least one of the following customized missions: a language practice mission, for example, repeating words displayed on a screen aloud; an emotional expression mission, for example, recording emotions felt after viewing a photo (e.g., a natural landscape) displayed on a display; a walking mission, for example, submitting a landscape photo after moving 100 meters using a walking aid; and an emotional stability mission, for example, expressing a brief appreciation after a religious activity (e.g., visiting a cathedral or temple).

Next, in case of Lewy body dementia, hallucinations, movement disorders, and sleep problems may occur frequently. Therefore, the computing device 600 may provide at least one of the following customized missions: a visual recognition mission, for example, finding a specific object (e.g., an animal) in a picture displayed on the screen and speaking about it; an exercise-related mission, for example, performing simple stretching movements and sending a completed photo; a music appreciation mission, for example, listening to a song displayed on the display through a speaker and speaking the title; and a stable activity mission, for example, coloring in a quiet environment (coloring book) and then submitting a photo.

Next, in case of frontotemporal dementia, decreased social interaction, emotional expression, and behavioral changes may occur. Accordingly, the computing device 600 may provide at least one of the following customized missions: a social conversation mission, for example, recording a conversation with a caregiver and then submitting voice data; an emotion recognition mission, for example, verbally describing facial expressions (e.g., laughter, sadness, etc.) displayed on the screen; a behavioral reinforcement mission, for example, submitting a photo after completing household chores (e.g., clearing the table); and a religious activity mission, for example, submitting a photo and impressions after visiting a religious facility.

Next, in case of Parkinson's disease, slow movement, language communication problems, and hand tremors may occur. Accordingly, the computing device 600 may provide at least one of the following customized missions: a simple movement mission, for example, rolling a ball with your hand and taking a photo; a language expression mission, for example, recording a conversation with a caregiver and then submitting it; a small muscle exercise mission, for example, folding paper (simple shapes) and then submitting a photo; and an auditory response mission, for example, matching the name of a sound displayed on the display.

Next, in case of mixed dementia, which is a combination of Alzheimer's disease and vascular dementia, the computing device 600 may provide at least one of the following customized missions: a memory and language mission, for example, recording the name and relationship while looking at a family photo; a walking and observation mission, for example, submitting a photo of surrounding objects after a 200-meter walk; a religion and emotion mission, for example, sharing thoughts after a religious activity (e.g., listening to a hymn); and a daily function mission, for example, pressing a complete button after taking medication.

Next, in case of alcoholic dementia, short-term memory problems and difficulty controlling emotions may occur. Therefore, the computing device 600 may provide at least one of the following customized missions: a memory stimulation mission, for example, speaking out loud a word displayed on the display after 10 seconds; an emotion management mission, for example, submitting a recording of emotions after listening to favorite music; a cognitive activity mission, for example, finding a nearby cafe and submitting a photo of the receipt; and a health management mission, for example, recording (taking a photo) and submitting a water intake record.

In addition, in cases of other types of dementia that do not fall under the examples described above, various symptoms may manifest depending on the specific cause. Therefore, the computing device 600 may provide customized missions, such as feature-linked missions, for example, actions tailored to the patient's specific symptoms (e.g., hand movements to reduce hand tremors), spatial recognition missions, for example, taking a photo of a specific location (e.g., a bench) in a nearby park, situational response missions, for example, finding the location of a dementia care center and following its route, and voice response missions, for example, simply answering a question displayed on a display (e.g., “How are you feeling today?”).

The customized missions exemplified for each dementia type may or may not have a priority. In the former case, after one customized mission is provided, another mission may be provided based on feedback.

As described above, the computing device 600 may generate and provide missions based on the dementia status.

For example, when creating a mission, the computing device 600 may consider the attributes of a dementia patient as described below.

When creating a mission, the mission is created using information related to the patient device 100, but other data described above may also be referenced.

First, in the case that a mission such as taking a landscape photo after a walk in a park is provided, the display in the first terminal 100 displays mission details and the park location, the GPS provides a path to the park through the route guidance module 160, and the camera may take and transmit park landscape photos through the photo shooting management module 170. In the second terminal 200, the mission progress confirmation module 250 may check the patient's movement status and photo transmission, the patient location management module 240 monitors in real time to ensure that the patient does not deviate from the path, and the patient location management module 620 in the computing device 600 records the walking path based on GPS data, and the report generation module 640 may generate a report based on the patient's movement and activity data.

When a mission to find a specific object (e.g., a red object) is provided, the display in the first terminal 100 displays information about the object required for the mission, the camera takes a picture of the red object and submits it to the photo shooting management module 170, the mission management module 210 in the second terminal 200 creates a mission and sets the object information, and the data sharing module 270 may share the photo data transmitted by the patient with the dementia care center. Meanwhile, the mission performance management module 670 in the computing device 600 analyzes the submitted data to determine whether the mission is successful, and the mission statistics generation module 680 may generate and record color recognition ability statistics.

In another mission, when taking a photo after visiting a pharmacy is provided, the GPS in the first terminal 100 may move to the pharmacy location through the route guidance module 160, the camera may take a photo of the pharmacy entrance and submit it to the photo shooting management module 170, the patient location management module 240 in the second terminal 200 may monitor the patient's movement status in real time, and the mission progress confirmation module 250 may perform mission completion processing by checking whether the photo has been submitted. Meanwhile, in the computing device 600, the emergency response module 630 may send a notification signal when an abnormal behavior occurs near the pharmacy, and the report generation module 340 may record and analyze the patient's pharmacy visit data.

In the case that another mission is provided to check the number of steps after walking 1 km, the accelerometer in the first terminal 100 measures the distance traveled and the number of steps and reflects them in the route guidance module 160, the display displays the number of steps and the distance traveled in real time, the patient status information module 230 in the second terminal 200 checks the patient's step count data in real time, and the point management module 280 may award points upon completion of the mission. Meanwhile, the mission statistics generation module 680 in the computing device 600 may collect distance traveled and number of steps data and generate physical activity statistics data.

In another mission, in the case that a mission such as sending a photo after a video call with a family member is provided, the microphone/speaker of the first terminal 100 analyzes the conversation during the video call through the voice analysis module 120, the camera takes and submits a family photo after the video call, the emergency contact support module 260 of the second terminal 200 maintains the video call with the patient, and the data sharing module 270 may transmit the call data and photo submission information to the dementia care center through the computing device 600. Meanwhile, the report generation module 640 of the computing device 600 may analyze and record the emotional interaction data of the dementia patient.

For another mission, in the case that a mission such as taking pictures after organizing specific items at home is provided, the display on the first terminal 100 may guide the user on what items need to be organized and how to organize them, and the camera may capture and transmit the organized state through the photo shooting management module 170. In the second terminal 200, the mission management module 210 may set the type of items and organizing criteria, and the mission progress confirmation module 250 may confirm the submitted photos and process the mission completion. Meanwhile, the mission performance management module 670 on the computing device 600 may analyze the organized state of the items and generate a report.

For another mission, in the case that a mission such as guessing the names of trees in a park is provided, the display on the first terminal 100 may provide pictures of trees and options, and the microphone may record the name of the selected tree through the voice analysis module 120. In the second terminal 200, the mission management module 210 may create missions and set options, and the data sharing module 270 may share selected data with the Dementia Care Center via the computing device 600. Meanwhile, the mission statistics generation module 680 in the computing device 600 may record and analyze plant recognition data.

Another mission, in the case that a mission such as purchasing items after visiting a traditional market is provided, the GPS in the first terminal 100 may navigate to the market location via the route guidance module 160, and the camera may capture and transmit photos of purchased items. In the second terminal 200, the patient location management module 240 may verify whether the dementia patient has visited the market, and the point management module 280 may award points upon successful completion of the mission. Meanwhile, in the computing device 600, the report generation module 640 may record market visit and purchase data.

In another mission, in the case that a mission such as recording ambient sounds is provided, the microphone in the first terminal 100 may record and submit ambient sounds through the voice analysis module 120, and the display may guide the recording status and progress. In the second terminal 200, the mission progress confirmation module 250 may manage the recording data confirmation and submission status. Meanwhile, the report generation module 640 in the computing device 600 may record environmental analysis data based on the recording data.

In another mission, in the case that a mission such as recording a daily diet and submitting photos is provided, the camera in the first terminal 100 may take and transmit photos of food before and after meals, and the display may check data regarding the mission status and submission status. In the second terminal 200, the mission progress confirmation module 250 may provide photo confirmation and feedback data, and the data sharing module 270 may share diet data with the dementia care center via the computing device 600. Meanwhile, in the computing device 600, the mission performance management module 670 may analyze the diet data and record the dementia patient's health status data.

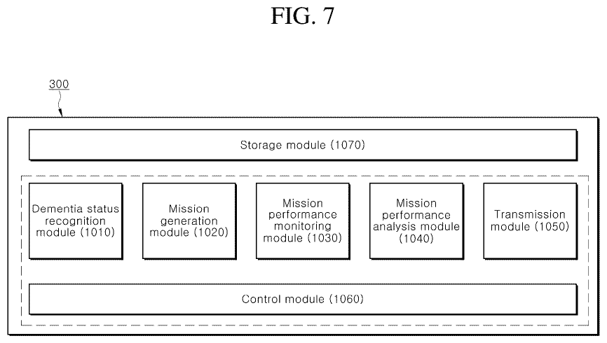

FIG. 7 illustrates an example of a dementia patient cognitive improvement device according to an embodiment of the present disclosure. The dementia patient cognitive improvement device of FIG. 7 may correspond to, or form part of, the computing device 600 of FIG. 6, for example. Therefore, for convenience, it will be described hereinafter as the computing device 600.

Referring to FIG. 7, the computing device 300 may largely include a processor and a storage module 1070. At this time, the processor may include a dementia status recognition module 1010, a mission generation module 1020, a mission performance monitoring module 1030, a mission performance analysis module 1040, a transmission module 1050, and a control module 1060. However, the storage module 1070 need not necessarily be an internal component of the computing device 300, and may be provided externally as long as it may be interoperable with the processor or computing device 300.

FIG. 7 describes from the perspective of the computing device 300, and reference may be made to the above-described content.

The dementia status recognition module 1010 may obtain the dementia status of a dementia patient, such as dementia attributes, dementia degree, dementia type, and other dementia-related information, and determine thereon, that is, recognize and determine the dementia status of the target dementia patient.

The mission generation module 1020 may generate mission data based on the dementia status of the target dementia patient, as recognized and determined by the dementia status recognition module 1010. The generated mission data may be transmitted to the second terminal 200 and/or the first terminal 100 via the transmission module 1050 under the control of the control module 1060.

The mission performance monitoring module 1030 collects and analyzes data for monitoring the dementia patient's mission performance based on the transmitted mission data. At this time, the data may be collected from the first terminal 100 and/or the second terminal 200.

The mission performance analysis module 1040 may analyze the dementia patient's mission performance based on the mission performance monitoring result.

The control module 1060 may control the overall operation of the processor or the computing device 600.

The storage module 1070 may temporarily store data collected, acquired, processed, and the like by the processor or the computing device 600 in connection with the present disclosure.

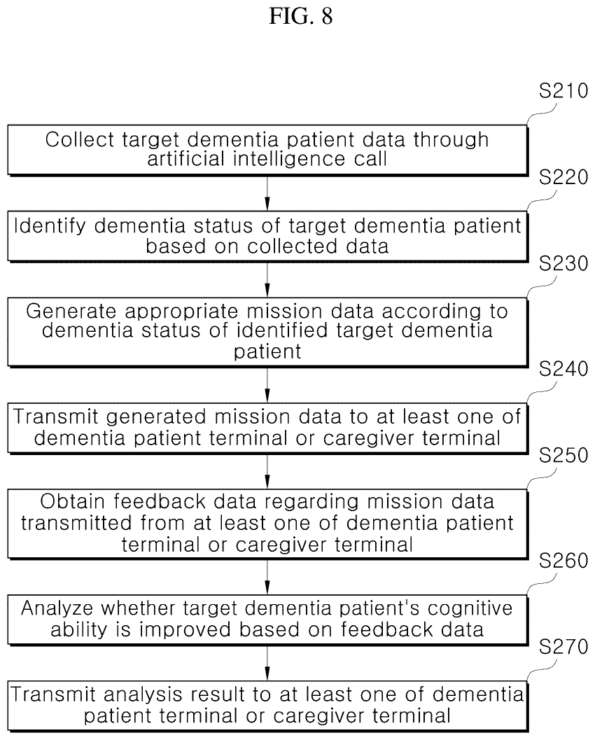

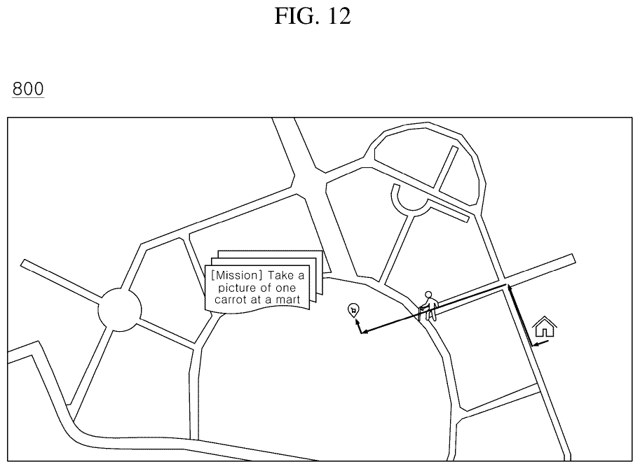

FIGS. 8 to 11 are flow charts illustrating a method for improving cognition of a dementia patient according to an embodiment of the present disclosure. FIG. 12 is an example of a user interface related to a mission provided to a dementia patient.

First, referring to FIG. 8, the computing device 300 may operate as follows.

The computing device 300 may collect target dementia patient data through an artificial intelligence call (step S210).

The computing device 300 may identify the dementia status of the target dementia patient based on the collected data (step S220).

The computing device 300 may generate appropriate mission data according to the dementia status of the identified target dementia patient (step S230).

The computing device 300 may transmit the generated mission data to at least one of the dementia patient terminal 100 or the caregiver terminal 200 (step S240).

The computing device 300 may obtain feedback data regarding the mission data transmitted from at least one of the dementia patient terminal 100 or the caregiver terminal 200 (step S250).

The computing device 300 may analyze whether the target dementia patient's cognitive ability is improved based on the feedback data (step S260).

The computing device 300 may transmit the analysis result to at least one of the dementia patient terminal 100 or the caregiver terminal 200 (step S270).

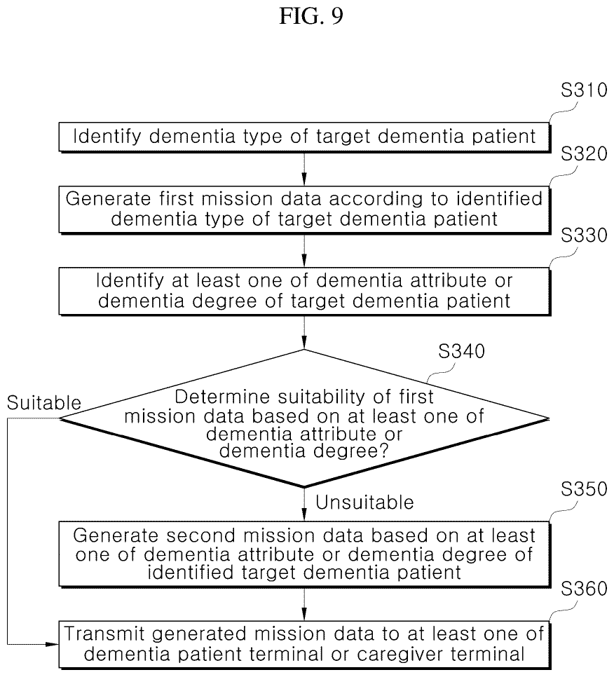

Next, referring to FIG. 9, the computing device 300 may perform the following operations.

The computing device 300 may identify the dementia type of the target dementia patient (step S310).

The computing device 300 may generate first mission data according to the identified dementia type of the target dementia patient (step S320).

The computing device 300 may further identify at least one of the dementia attribute or the dementia degree of the target dementia patient (step S330).

The computing device 300 may determine the suitability of the first mission data based on at least one of the dementia attribute or the dementia degree (step S340).

In the case that the first mission data is determined to be inappropriate based on the suitability determination result, the computing device 300 may generate second mission data based on at least one of the dementia attribute or the dementia degree of the identified target dementia patient (step S350).

The computing device 300 may transmit the generated mission data to at least one of the dementia patient terminal 100 or the caregiver terminal 200 (step S360).

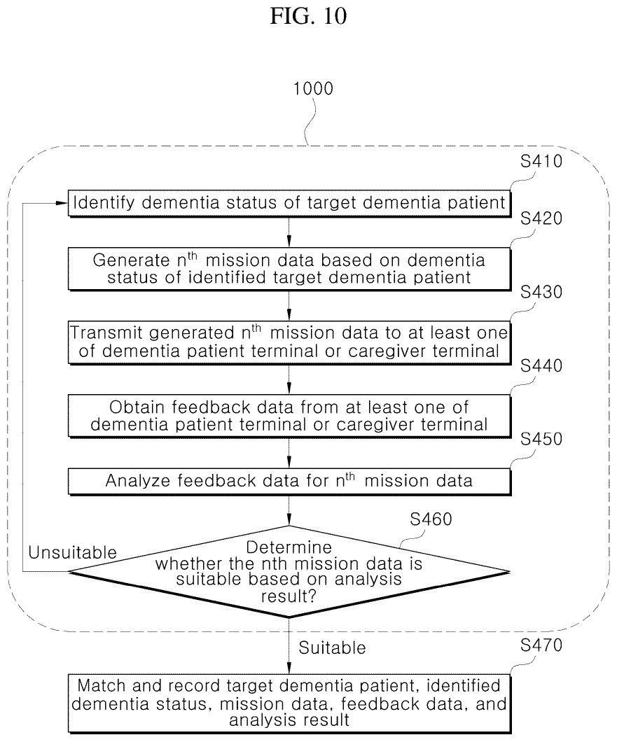

Next, referring to FIG. 10, the computing device 300 may operate as follows.

The computing device 300 may identify the dementia status of the target dementia patient (step S410).

The computing device 300 may generate n^{th} mission data (where n is a positive integer) based on the dementia status of the identified target dementia patient (step S420).

The computing device 300 may transmit the generated n^{th} mission data to at least one of the dementia patient terminal 100 or the caregiver terminal 200 (step S430).

The computing device 300 may obtain feedback data from at least one of the dementia patient terminal 100 or the caregiver terminal 200 (step S440).

The computing device 300 may analyze feedback data for the n^{th} mission data (step S450).

The computing device 300 may determine whether the n^{th} mission data is suitable based on the analysis result (step S460).

At this time, in the case that the computing device 300 determines that the n^{th} mission data is not suitable, the computing device 300 may re-execute the process 1000 from step S410 or S420. This process may be repeated repeatedly until suitable mission data is found.

The computing device 300 may match and record the target dementia patient, the identified dementia status, the mission data, the feedback data, and the analysis result (step S470).

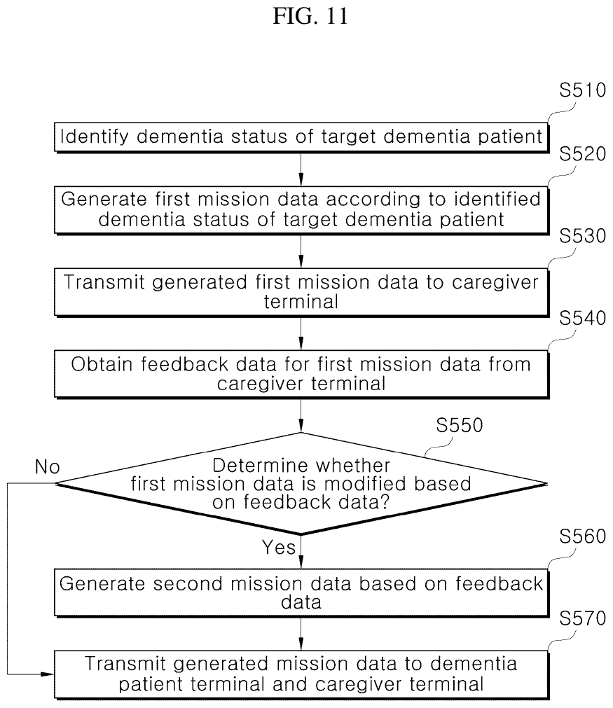

Finally, referring to FIG. 11, the computing device 300 may operate as follows.

The computing device 300 may identify the dementia status of the target dementia patient (step S510).

The computing device 300 may generate the first mission data according to the identified dementia status of the target dementia patient (step S520).

The computing device 300 may transmit the generated first mission data to the caregiver terminal 200 (step S530).

The computing device 300 may obtain the feedback data for the first mission data from the caregiver terminal 200 (step S540).

The computing device 300 may determine whether the first mission data is modified based on the feedback data (step S550).

As a result of the determination in step S550, in the case that the feedback data obtained by the caregiver terminal 200 is modified, the computing device 300 may generate second mission data based on the feedback data (step S560).

The computing device 300 may transmit the generated mission data to the dementia patient terminal 100 and the caregiver terminal 200 (step S570).

FIG. 12 illustrates an example of a user interface 3200 provided to the first or second terminal 100 or 200 when the mission data such as “take a picture of one carrot at a mart” is generated.

The brief descriptions in FIGS. 8 to 11 may refer to the contents described in at least one of FIGS. 1 to 7 described above. The sequences or steps illustrated in FIGS. 8 to 11 described above may operate differently from the illustrated sequences. Furthermore, at least some of the sequences or steps in FIGS. 8 to 11 may be combined or linked with the sequences or steps in other drawings.

The steps of the method or algorithm described in connection with the embodiments of the present disclosure may be implemented directly in hardware, implemented as a software module executed by hardware, or implemented by a combination thereof. The software module may reside in a random access memory (RAM), a read only memory (ROM), an erasable programmable ROM (EPROM), an electrically erasable programmable ROM (EEPROM), a flash memory, a hard disk, a removable disk, a CD-ROM, or any other form of computer-readable recording medium well known in the art to which the present disclosure pertains.

While the embodiments of the present disclosure have been described above with reference to the accompanying drawings, those skilled in the art will appreciate that the present disclosure may be implemented in other specific forms without changing the technical spirit or essential characteristics thereof. Therefore, it should be understood that the embodiments described above are illustrative in all respects and not restrictive.

According to the present disclosure, the effect of improving dementia cognitive status can be maximized by providing customized mission data according to the dementia status of the target dementia patient.

What is claimed is:

1. A device for improving cognition of a dementia patient, comprising:
a memory configured to store various mission data; and
a processor configured to communicate with the memory and exchanging data,
wherein the processor is configured to:
conduct a dementia test on the dementia patient through an artificial intelligence call and collect a result of the dementia test,
obtain a dementia status of the dementia patient based on the result of the dementia test, wherein the dementia status includes a dementia attribute, a dementia degree, and a dementia type,
select first mission data for improving cognition of the dementia patient based on the dementia status, and transmit the first mission data to at least one of a dementia patient terminal or a caregiver terminal,
receive feedback data on the first mission data from at least one of the dementia patient terminal or the caregiver terminal,
when conducting the dementia test, analyze usage patterns of applications installed on the dementia patient terminal and selectively provide a type of the dementia test, and based on a use frequency of a news application of the dementia patient being high of among the applications, conduct the dementia test on topics related to current affairs, and
collect location information of the dementia patient terminal to identify a frequency of going out or a change in location of the dementia patient, and determine the dementia patient's behavior based on a determined result to adjust a difficulty of the dementia test related to cognitive function,
wherein the attribute includes an age, a gender, an education level, a residence, a hometown, a presence of spouse, an income level, a presence of allergy, and a use of walking aid, and
wherein the processor is further configured to:
when selecting the first mission data, select a mission that reflects the interests and activity preferences of the gender,
select the mission considering a problem type and a difficulty level based on the education level,
select the mission that reflects environmental differences between urban and rural areas based on the residence,
select the mission that stimulates memories related to the hometown of the dementia patient based on the hometown,
select the mission that is performed with the spouse based on the presence of the spouse,
select the mission considering a cost required to perform the mission based on the income level,
select the mission to avoid an allergen while performing the mission based on the allergy,
select the mission to limit a movement distance of the dementia patient and an activity type based on the use of walking aid,
based on the dementia type being Alzheimer's disease, select the mission that stimulates memory,
based on the dementia type being vascular dementia, select the mission that repeats words presented on a screen and the mission that expresses emotions,
based on the dementia type being Lewy body dementia, select the visual recognition mission to identify and identify a specific object in a displayed image or the mission to listen to music and then state a title,
based on the dementia type being frontotemporal dementia, select at least one mission to record a conversation with the caregiver and then submit voice data, the mission to verbally describe facial expression displayed on the screen, and the mission to submit a photo after completing a housework, and
based on the dementia type being Parkinson's disease dementia, select at least one of the mission of rolling a ball with a hand and taking a picture, the mission of recording a conversation with the caregiver and submitting the record, or the mission of submitting a picture after small muscle exercise.

2. The device of claim 1,
wherein the processor is configured to:
analyze whether the dementia patient's cognitive improvement is present based on the feedback data, and transmit the analysis result to at least one of the dementia patient terminal or the caregiver terminal.

3. The device of claim 1,
wherein the processor is configured to determine, based on the feedback data, whether the first mission data is appropriate for the dementia patient.

4. The device of claim 1,
wherein the processor is configured to:
based on the feedback data including a request for modification of the first mission data, generate and provide second mission data based on the feedback data.

5. The device of claim 4,
wherein the processor is configured to:
based on the feedback data including information for a performance of the first mission data, provide a point according to a degree of mission performance and completion.

6. The device of claim 5,
wherein the dementia type, the dementia attribute, and the dementia degree included in the dementia status are assigned different priorities or weight values during the first or second mission data generation process.

7. A method for improving cognition of a dementia patient, performed by an electronic device, comprising:
conducting a dementia test on the dementia patient through an artificial intelligence call and collecting a result of the dementia test;
obtaining a dementia status of the dementia patient based on the result of the dementia test, wherein the dementia status includes a dementia attribute, a dementia degree, and a dementia type;
selecting first mission data for improving cognition of the dementia patient based on the dementia status, and transmitting the first mission data to at least one of a dementia patient terminal or a caregiver terminal;
receiving feedback data on the first mission data from at least one of the dementia patient terminal or the caregiver terminal;
when conducting the dementia test, analyzing usage patterns of applications installed on the dementia patient terminal and selectively providing a type of the dementia test, and based on a use frequency of a news application of the dementia patient being high of among the applications, conduct the dementia test on topics related to current affairs; and
collecting location information of the dementia patient terminal to identify a frequency of going out or a change in location of the dementia patient, and determine the dementia patient's behavior based on a determined result to adjust a difficulty of the dementia test related to cognitive function,
wherein the attribute includes an age, a gender, an education level, a residence, a hometown, a presence of spouse, an income level, a presence of allergy, and a use of walking aid, and
wherein the electronic device is configured to:
when selecting the first mission data, select a mission that reflects the interests and activity preferences of the gender,
select the mission considering a problem type and a difficulty level based on the education level,
select the mission that reflects environmental differences between urban and rural areas based on the residence,
select the mission that stimulates memories related to the hometown of the dementia patient based on the hometown,
select the mission that is performed with the spouse based on the presence of the spouse,
select the mission considering a cost required to perform the mission based on the income level,
select the mission to avoid an allergen while performing the mission based on the allergy,
select the mission to limit a movement distance of the dementia patient and an activity type based on the use of walking aid,
based on the dementia type being Alzheimer's disease, select the mission that stimulates memory,
based on the dementia type being vascular dementia, select the mission that repeats words presented on a screen and the mission that expresses emotions,
based on the dementia type being Lewy body dementia, select the visual recognition mission to identify and identify a specific object in a displayed image or the mission to listen to music and then state a title,
based on the dementia type being frontotemporal dementia, select at least one mission to record a conversation with the caregiver and then submit voice data, the mission to verbally describe facial expression displayed on the screen, and the mission to submit a photo after completing a housework, and
based on the dementia type being Parkinson's disease dementia, select at least one of the mission of rolling a ball with a hand and taking a picture, the mission of recording a conversation with the caregiver and submitting the record, or the mission of submitting a picture after small muscle exercise.

8. A system for improving cognition of a dementia patient, comprising:
a dementia patient terminal configured to provide mission data and obtain data related to perform a mission;
a caregiver terminal configured to select or modify the mission data to be provided from the terminal of the dementia patient; and
a computing device configured to provide mission data to at least one of the dementia patient terminal or the caregiver terminal,
wherein the computing device is configured to:
conduct a dementia test on the dementia patient through an artificial intelligence call and collect a result of the dementia test,
obtain a dementia status of the dementia patient based on the result of the dementia test, wherein the dementia status includes a dementia attribute, a dementia degree, and a dementia type,
select first mission data for improving cognition of the dementia patient based on the dementia status, and transmit the first mission data to at least one of a dementia patient terminal or a caregiver terminal,
receive feedback data on the first mission data from at least one of the dementia patient terminal or the caregiver terminal,
when conducting the dementia test, analyze usage patterns of applications installed on the dementia patient terminal and selectively provide a type of the dementia test, and based on a use frequency of a news application of the dementia patient being high of among the applications, conduct the dementia test on topics related to current affairs, and
collect location information of the dementia patient terminal to identify a frequency of going out or a change in location of the dementia patient, and determine the dementia patient's behavior based on a determined result to adjust a difficulty of the dementia test related to cognitive function,
wherein the attribute includes an age, a gender, an education level, a residence, a hometown, a presence of spouse, an income level, a presence of allergy, and a use of walking aid, and
wherein the computing device is further configured to:
when selecting the first mission data, select a mission that reflects the interests and activity preferences of the gender,
select the mission considering a problem type and a difficulty level based on the education level,
select the mission that reflects environmental differences between urban and rural areas based on the residence,
select the mission that stimulates memories related to the hometown of the dementia patient based on the hometown,
select the mission that is performed with the spouse based on the presence of the spouse,
select the mission considering a cost required to perform the mission based on the income level,
select the mission to avoid an allergen while performing the mission based on the allergy,
select the mission to limit a movement distance of the dementia patient and an activity type based on the use of walking aid,
based on the dementia type being Alzheimer's disease, select the mission that stimulates memory,
based on the dementia type being vascular dementia, select the mission that repeats words presented on a screen and the mission that expresses emotions,
based on the dementia type being Lewy body dementia, select the visual recognition mission to identify and identify a specific object in a displayed image or the mission to listen to music and then state a title,
based on the dementia type being frontotemporal dementia, select at least one mission to record a conversation with the caregiver and then submit voice data, the mission to verbally describe facial expression displayed on the screen, and the mission to submit a photo after completing a housework, and
based on the dementia type being Parkinson's disease dementia, select at least one of the mission of rolling a ball with a hand and taking a picture, the mission of recording a conversation with the caregiver and submitting the record, or the mission of submitting a picture after small muscle exercise.
